(19) United States
(12) Patent Application Publication
(10) Pub. No.: US 2013/0225671 A1
(43) Pub. Date: Aug. 29, 2013

(54) Methods of Treating Seizure Disorders

(71) Applicant: Massachusetts Institute of Technology, (US)

(72) Inventors: Mark F. Bear, Cambridge, MA (US); Emily Osterweil, Cambridge, MA (US)

(73) Assignee: Massachusetts Institute of Technology, Cambridge, MA (US)

(21) Appl. No.: 13/862,123

(22) Filed: Apr. 12, 2013

Related U.S. Application Data
(63) Continuation of application No. PCT/US2011/057099, filed on Oct. 20, 2011.
(60) Provisional application No. 61/405,446, filed on Oct. 21, 2010.

(51) Int. Cl. A61K 31/366

(52) U.S. Cl. CPC A61K 31/366
USPC 514/460

(57) ABSTRACT

Seizure disorders are treated by Ras inhibitors. In an embodiment, the Ras inhibitor includes a farnesyl transferase inhibitor. In another embodiment, the farnesyl transferase inhibitor includes a 3-hydroxy-3-methylglutaryl-Coenzyme A reductase inhibitor. In another embodiment, the 3-hydroxy-3-methylglutaryl-Coenzyme A reductase inhibitor is not a Ras inhibitor. Subjects having fragile X syndrome, autism, Angelman syndrome, Costello syndrome, cardio facio cutaneous syndrome, neurofibromatosis type I, Noonan syndrome and Coffin-Lowry syndrome that have seizure disorders can be treated.

### RELATED APPLICATIONS

This application is a continuation of International Application No. PCT/US2011/057099, which designated the United States and was filed on Oct. 20, 2011, published in English, claims the benefit of U.S. Provisional Application No. 61/405,446, filed on Oct. 21, 2010. The entire teachings of the above applications are incorporated herein by reference.

### GOVERNMENT SUPPORT

This invention was made with government support under HD046943 and R21MH090452 awarded by the National Institutes of Health. The government has certain rights in the invention.

### BACKGROUND OF THE INVENTION

Seizure disorders, including epilepsy, can affect daily activities of adults and children. Seizures can develop in early childhood or adolescence. Seizure disorders can be associated with abnormal electrical activity in the brain resulting in temporary loss of consciousness, body convulsions, unusual movements and staring spells, all of which affect daily activities and the health of the affected individual. Current treatments for seizure disorders, including seizure disorders in individuals with autism, fragile X syndrome, Angelman syndrome, Costello syndrome, cardio facio cutaneous syndrome (CFC), neurofibromatosis type I, Noonan syndrome and Coffin-Lowry syndrome, can include drug therapy, such as anticonvulsant drugs. However, such treatments can result in unwanted side-effects and may not treat the underlying cause of the seizure disorder.

### SUMMARY OF THE INVENTION

The invention is generally directed to methods of treating a seizure disorder in an individual by administering compositions that include a Ras inhibitor and/or an 3-hydroxy-3-methylglutaryl-Coenzyme A reductase inhibitor.

In an embodiment, the invention is a method of treating a subject having a seizure disorder, comprising the step of administering a composition that includes at least one Ras inhibitor.

In another embodiment, the invention is a method of treating a subject having a seizure disorder, comprising the step of administering a composition that includes at least one 3-hydroxy-3-methylglutaryl-Coenzyme A reductase inhibitor.

The methods of the invention have the advantage of normalizing synaptic signaling in the central nervous system to thereby treat an underlying cause of the seizure disorder.

### BRIEF DESCRIPTION OF THE DRAWINGS

FIGS. 1A-1I depict changes in protein synthesis and ERK1/2 activation in hippocampal slices.

FIGS. 1A-1C show that protein synthesis is excessive in the Fmr1 KO.

FIGS. 1D-1F show that the MEK1/2-ERK1/2 inhibitor U0126 corrects excessive protein synthesis and reduces ERK1/2 activation in the Fmr1 KO.

FIGS. 1G-1I shows that LOVASTATIN (Formula I, “lovastn”) in the lactone form can correct protein synthesis and reduce ERK1/2 activation in the Fmr1 KO.

FIGS. 2A-2G show changes in the presence of LOVASTATIN (Formula I, “lovastn”) in the acid form.

FIGS. 2A-C show that Formula I corrects protein synthesis in Fmr1 KO hippocampal slices. The significant normalization of protein synthesis by Formula I is shown as a summary bar graph (FIG. 2A) and yoked WT-KO slices with vehicle (FIG. 2B) and with 50 μM Formula I (FIG. 2C).

FIGS. 2D and 2E show that Formula I inhibits Ras activation in hippocampal slices.

FIGS. 2F and 2G show that Formula I downregulates ERK1/2 in hippocampal slices. Error bars=s.e.m.

FIGS. 3A-3L show that Formula I (LOVASTATIN, “lov”) significantly reduces AGS incidence and severity in the Fmr1 KO on the C57BL6 and FVB backgrounds. Fmr1 KO and WT mice on the C57BL6 (FIGS. 3A-3F) or FVB (FIGS. 3G-3L) backgrounds were treated as indicated, tested for AGS, and scored for wild running, clonic seizure, tonic seizure and death.

FIG. 3A shows that injection of 30 mg/kg of Formula I acid significantly reduces AGS incidence.

FIGS. 3B and 3C show that injection of 30 mg/kg of Formula I acid attenuates AGS severity in Fmr1 KO mice.

FIG. 3D shows that injection of 100 mg/kg Formula I significantly reduces AGS incidence and (FIGS. 3E and 3F) attenuates AGS severity in Fmr1 KO mice on the C57BL6 background.

FIGS. 3E and 3F show that injection of 100 mg/kg Formula I attenuates AGS severity in Fmr1 KO mice on the C57BL6 background.

FIG. 3G shows that injection of 30 mg/kg Formula I does not significantly reduce AGS incidence or (FIGS. 3H and 3I) severity in Fmr1 KO mice on the FVB background.

FIGS. 3H and 3I show that injection of 30 mg/kg Formula I does not significantly reduce AGS severity in Fmr1 KO mice on the FVB background.

FIG. 3J shows that injection of 100 mg/kg Formula I significantly reduces AGS incidence and (FIGS. 3K and 3L) severity in Fmr1 KO mice on the FVB background.

FIGS. 3K and 3L show that injection of 100 mg/kg Formula I significantly reduces AGS severity in Fmr1 KO mice on the FVB background.

FIGS. 4A-4D show that Formula I lactone and oral administration significantly reduces AGS incidence and severity in the Fmr1 KO. Fmr1 KO and WT mice (C57BL6) were treated as indicated and tested for AGS. The following stages of AGS severity were scored: wild running, clonic seizure, tonic seizure, and death.

FIG. 4A shows that injection of 30 mg/kg Formula I lactone significantly reduces AGS incidence and severity in Fmr1 KO mice.

FIGS. 4B and 4C show that injection of 30 mg/kg Formula I lactone significantly reduces AGS severity in Fmr1 KO mice.

FIG. 4D shows that 48 h feeding of 0.1% Formula I chow significantly reduces AGS incidence in Fmr1 KO mice.

FIGS. 4E and 4F show that 48 h feeding of 0.1% Formula I chow significantly reduces AGS severity in Fmr1 KO mice.

### DETAILED DESCRIPTION OF THE INVENTION

A description of example embodiments of the invention follows. The features and other details of the invention, either as steps of the invention or as combinations as parts of the invention, will now be particularly described and pointed out in the claims. It will be understood that the particular embodiments of the invention are shown by way of illustration and not as limitations of the invention. The principle features of this invention can be employed in various embodiments without departing from the scope of the invention.

In an embodiment, the invention is a method of treating a subject having a seizure disorder, comprising the step of administering a composition that includes at least one Ras inhibitor.

In another embodiment, the invention is a method of treating a subject having a seizure disorder, comprising the step of administering a composition that includes at least one 3-hydroxy-3-methylglutaryl-Coenzyme A reductase inhibitor.

Ras (RAt Sarcoma) proteins (also referred to herein as “Ras”) are guanosine-nucleotide-binding proteins (G-proteins) involved in all signaling. Genes encoding Ras proteins were initially identified as oncogenes. Ras is associated with the plasma membrane of a cell because of prenylation and palmitoylation or the combination of prenylation and a polybasic sequence adjacent to the prenylation site. The carboxy-terminal CaaX amino acid sequence of Ras is initially farnesylated at a cysteine residue in the cytoplasm of the cell, which permits Ras to loosely insert into the endoplasmatic reticulum and other cellular membranes. The aaX tripeptide of Ras is then cleaved from the carboxy-terminus of Ras by a prenyl-protein specific endoprotease and the resulting carboxy-terminus methylated by a methyltransferase. Electrostatic interactions between the positively charged basic sequence of Ras with negative charges at the inner surface of the plasma membrane account for the predominant localization of Ras at the cell surface.

“Ras inhibitor,” as used herein, means a compound that prevents activation of a Ras protein. In an embodiment, a Ras inhibitor can bind Ras, thereby preventing Ras from hydrolying GTP (guanosine triphosphate) and, thus, preventing activation of downstream signaling pathways.

The carboxy-terminal membrane targeting region of Ras has an aaX motif that is modified by farnesyl transferase resulting in the addition of a 15-carbon isoprenoid (farnesyl group). In an embodiment, the Ras inhibitor is a farnesyl transferase inhibitor.

“Farnesyl transferase inhibitor,” as used herein, mean that the Ras inhibitor prevents the addition of a farnesyl group to Ras.

In an embodiment, the farnesyl transferase inhibitor is a 3-hydroxy-3-methylglutaryl-Coenzyme A reductase inhibitor (also referred to herein as “statins”).

“A 3-hydroxy-3-methylglutaryl-Coenzyme A reductase inhibitor,” as used herein, mean that the compound prevents the enzymatic activity of 3-hydroxy-3-methylglutaryl-Coenzyme A reductase. In an embodiment, the 3-hydroxy-3-methylglutaryl-Coenzyme A reductase inhibitor inhibits cell signaling via Ras. In another embodiment, the 3-hydroxy-3-methylglutaryl-Coenzyme A reductase inhibitor prevents cell signaling through pathways other than Ras and is not a Ras inhibitor. For example, the 3-hydroxy-3-methylglutaryl-Coenzyme A reductase inhibitor can inhibit glycogen synthase kinase 3beta (GSK3beta) signaling.

Statins are compounds that low cholesterol levels by inhibiting the enzyme HMG (3-hydroxy-3-methyl-glutaryl)-Coenzyme A (also referred to herein as “CoA”) reductase. HMG-CoA reductase is the rate-controlling enzyme of the mevalonate pathway, which produces cholesterol and other isoprenoids. In mammalian cells, HMG-CoA reductase is normally suppressed by cholesterol derived from the internalization and degradation of low density lipoprotein (LDL) by the LDL receptor and oxidized species of cholesterol. Competitive inhibitors of HMG-CoA reductase induce the expression of LDL receptors in the liver, which, in turn, increase the catabolism of plasma LDL and lower the plasma concentration of cholesterol. HMG-CoA reductase is anchored in the membrane of the endoplasmic reticulum in the cytoplasm of cells. In an embodiment, the 3-hydroxy-3-methylglutaryl-Coenzyme A reductase inhibitor can include at least one member selected from the group consisting of Formulas I and II:

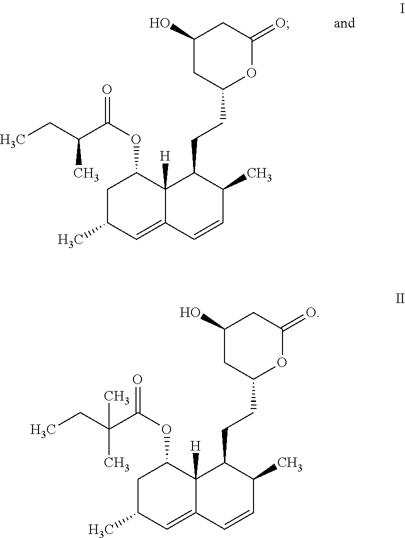 [SMILES: CC[C@H](C)C(=O)O[C@H]1C[C@@H](C)C=C2C=C[C@H](C)[C@H](CC[C@@H]3C[C@@H](O)CC(=O)O3)[C@H]21 ; "and" ; CCC(C)(C)C(=O)O[C@H]1C[C@@H](C)C=C2C=C[C@H](C)[C@H](CC[C@@H]3C[C@@H](O)CC(=O)O3)[C@H]21]

In another embodiment, the invention is a method of treating a seizure disorder in a subject comprising administering a composition that includes at least one of a MEK inhibitor or an ERK (extracellular signal-regulated kinase) inhibitor (also referred to as a “MAPK (mitogen-activated protein kinase) inhibitor”).

Ras, when activated, activates RAF kinase, a protein kinase that phosphorylates and activates MEK (MEK1 and MEK2). Phosphorylated MEK activates ERK, which, in turn, alters cellular processes.

A “MEK inhibitor,” as used herein, means a compound that prevents activation of MEK. Exemplary MEK inhibitors for use in the methods include XL518 ([3,4-difluoro-2-[(2-fluoro-4-iodophenyl)amino]phenyl][3-hydroxy-3-[(2R)-2-piperidinyl]-1-azetidinyl]methane; EXELIXIS), CI-1040 (2-(2-chloro-4-iodophenylamino)-N-(cyclopropylmethoxy)-3,4-difluorobenzamide); CAS No: 212631-79-3; MEDKOO BIOSCIENCE), PD0325901 (N-[(2R)-2,3-dihydroxypropoxy]-3,4-difluoro-2-[(2-fluoro-4-iodophenyl)amino]-benzamide); CAS No: 391210-10-9, CAYMAN CHEMICAL), SELUMETINIB (C₁₇H₁₅BrClFN₄O₃, CAS No.: 606143-52-6, SELLECKCHEM), GSK1120212 (N-[3-[3-cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]acetamide); CAS No: 871700-17-3, ACTIVE BIOCHEM), AS703026 ((S)—N-(2,3-dihydroxypropyl)-3-((2-fluoro-4-iodophenyl)amino)isonicotinamide); CAS No: 1236699-92-5, SELLECKCHEM), AZD8330 (2-((2-fluoro-4-iodophenyl)amino)-N-(2-hydroxyethoxy)-1,5-dimethyl-6-oxo-1,6-dihydropyridine-3-carboxamide); CAS No.: 869357-68-6, SELLECKCHEM), PD318088 (5-bromo-N-(2,3-dihydroxypropoxy)-3,4-difluoro-2-[(2-fluoro-4-iodophenyl)amino]benzamide); CAS No: 391210-00-7; SELLECKCHEM), PD98059 (2-(2-amino-3-methoxyphenyl)-4H-1-benzopyran-4-one), CAS No: 167869-21-8, REAGENTS DIRECT), U0126 (PROMEGA), TAK-733 (8-methylpyrido[2,3-d]pyrimidine-4,7(3H,8H)-dione); CAS No.: 1035555-63-5, SELLECKCHEM), ARRY-162 (NOVARTIS), ARRY-300 (NOVARTIS), PD184161 (5-bromo-2-[(2-chloro-4-iodophenyl)amino]-N-(cyclopropylmethoxy)-3,4-difluoro-benzamide); CAS No: 212631-67-9, CAYMAN CHEMICAL), and SL327 (alpha-[amino[(4-aminophenyl)thio]methylene]-2-(trifluoromethyl)benzeneacetonitrile); CAS No. 305350-87-2, TO C R1S). The chemical structures of exemplary MEK inhibitors appear in Table 1.

TABLE 1
Chemical structures of exemplary MEK inhibitors.

| IUPAC Name | Chemical Structure |
|---|---|
| [3,4-difluoro-2-[(2-fluoro-4- iodophenyl)amino]phenyl][3-hydroxy-3- [(2R)-2-piperidinyl]-1- azetidinyl]methane | 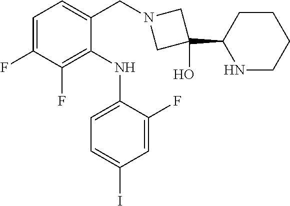 [SMILES: OC1([C@H]2CCCCN2)CN(Cc2ccc(F)c(F)c2Nc2ccc(I)cc2F)C1] |
| 2-(2-chloro-4-iodophenylamino)-N- (cyclopropylmethoxy)-3,4- difluorobenzamide | 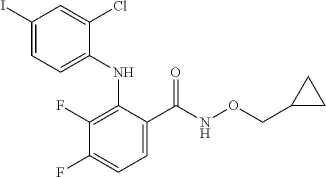 [SMILES: O=C(NOCC1CC1)c1ccc(F)c(F)c1Nc1ccc(I)cc1Cl] |
| N-[(2R)-2,3-dihydroxypropoxy]-3,4- difluoro-2-[(2-fluoro-4- iodophenyl)amino]-benzamide | 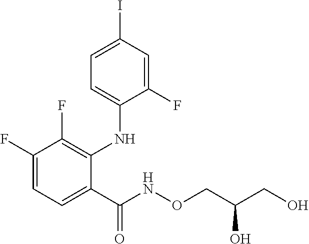 [SMILES: O=C(NOC[C@H](O)CO)c1ccc(F)c(F)c1Nc1ccc(I)cc1F] |
| 6-(4-bromo-2-chloroanilino)-7-fluoro-N- (2-hydroxyethoxy)-3- methylbenzimidazole-5-carboxamide | 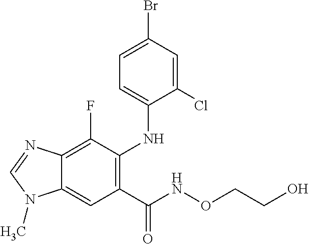 [SMILES: Cn1cnc2c(F)c(Nc3ccc(Br)cc3Cl)c(C(=O)NOCCO)cc21] |
| N-[3-[3-cyclopropyl-5-[(2-fluoro-4- iodophenyl)amino]-3,4,6,7-tetrahydro- 6,8-dimethyl-2,4,7-trioxopyrido[4,3- d]pyrimidin-1(2H)-yl]phenyl]acetamide | 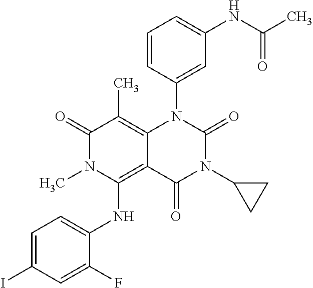 [SMILES: CC(=O)Nc1cccc(-n2c(=O)n(C3CC3)c(=O)c3c(Nc4ccc(I)cc4F)n(C)c(=O)c(C)c32)c1] |
| (S)-N-(2,3-dihydroxypropyl)-3-((2- fluoro-4- iodophenyl)amino)isonicotinamide | 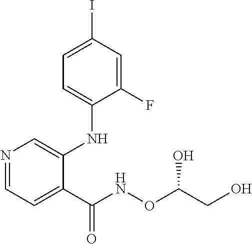 [SMILES: O=C(NO[C@H](O)CO)c1ccncc1Nc1ccc(I)cc1F] |
| 2-((2-fluoro-4-iodophenyl)amino)-N-(2- hydroxyethoxy)-1,5-dimethyl-6-oxo-1,6- dihydropyridine-3-carboxamide | 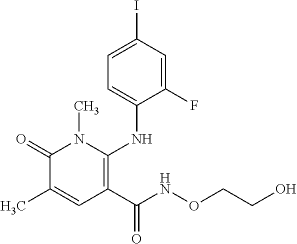 [SMILES: Cc1cc(C(=O)NOCCO)c(Nc2ccc(I)cc2F)n(C)c1=O] |
| 5-bromo-N-(2,3-dihydroxypropoxy)-3,4- difluoro-2-[(2-fluoro-4- iodophenyl)amino]benzamide | 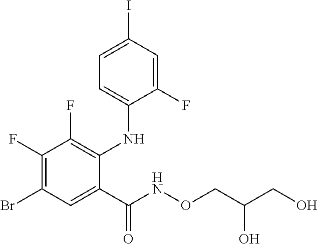 [SMILES: O=C(NOCC(O)CO)c1cc(Br)c(F)c(F)c1Nc1ccc(I)cc1F] |
| 2-(2-amino3-methoxyphenyl)-4H-1- benzopyran-4-one | 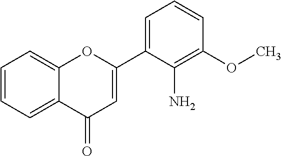 [SMILES: COc1cccc(-c2cc(=O)c3ccccc3o2)c1N] |
| 8-methylpyrido[2,3-d]pyrimidine- 4,7(3H,8H)-dione | 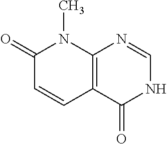 |
| 5-bromo-2-[(2-chloro-4- iodophenyl)amino]-N- (cyclopropylmethoxy)-3,4-difluoro- benzamide | 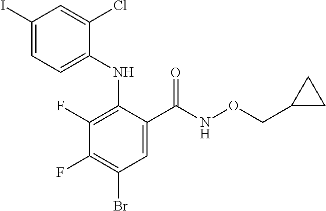 [SMILES: O=C(NOCC1CC1)c1cc(Br)c(F)c(F)c1Nc1ccc(I)cc1Cl] |
| alpha-[amino[(4- aminophenyl)thio]methylene]-2- (trifluoromethyl)benzeneacetonitrile | 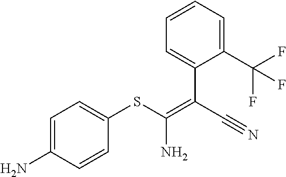 [SMILES: N#C/C(=C(\N)Sc1ccc(N)cc1)c1ccccc1C(F)(F)F] |

The MEK inhibitor ARRY-162 (NOVARTIS) has a MEK Enzyme IC₅₀=12 nM; Malme-3M pERK IC₅₀=11 nM; Human Whole Blood TPA-induced TNF-α=23 nM, IL-1β and IL-6=21 nM. Safety data for ARRY-162 is as follows: Genotoxicity: Negative; Cytochrome p450 Inhibition-IC50: >25 μM (5 major isoforms); Kinase selectivity screen has no activity against ˜200 other kinases at 1 μM; and receptor screen is clean against 25 receptors. In vivo efficacy of ARRY-162—Rat carrageenan paw edema: ED₅₀˜7 mg/kg, PO, once; Rat Collagen-Induced Arthritis: ED₅₀=3 mg/kg, PO, QD×7; and rat adjuvant-induced arthritis: ED₅₀=10 mg/kg, PO, QD×7 (Winkler, J., et al., Inflamm. Res. Supplement 3: FC11.4 (2007); Winkler, J., et al., 9^{th} Annual World Congress of Inflammation, July 2009, Tokyo, JP WS06-15).

An “ERK inhibitor,” as used herein, means a compound that prevents activation of ERK. Exemplary ERK inhibitors for use in the methods of the invention include FR180204 (5-(2-phenylpyrazolo[1,5-a]pyridin-3-yl)-1H-pyrazolo[3,4-c]pyridazin-3-amine), CAS No: 865362-74-9, SANTA CRUZ BIOTECHNOLOGY), and CAY10561 N-[1-(3-chloro-4-fluorophenyl)-2-hydroxyethyl]-4-[4-(3-chlorophenyl)-1H-pyrazol-3-yl]-1H-pyrrole-2-carboxamide; CAS No: 933786-58-4, CAYMAN CHEMICAL), and AEZS-131 (AETERNA ZENTARIS). In another embodiment, the Ras inhibitor (e.g., farnesyl transferase inhibitor, 3-hydroxy-3-methylglutaryl-Coenzyme A reductase inhibitor) can be administered to a subject having a seizure disorder in combination with at least one compound that down regulates Group I mGluR signaling. The compound that down regulates Group I mGluR signaling can be administered to the subject before, concomitant with or after administration of the Ras inhibitor. The chemical structures of exemplary ERK inhibitors appear in Table 2.

TABLE 2
Chemical structures of exemplary ERK inhibitors.

| IUPAC Name | Chemical Structure |
|---|---|
| 5-(2-phenylpyrazolo[1,5-a]pyridin-3-yl)- 1H-pyrazolo[3,4-c]pyridazin-3-amine | 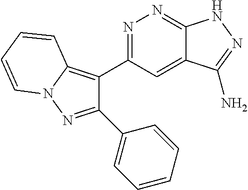 [SMILES: Nc1n[nH]c2nnc(-c3c(-c4ccccc4)nn4ccccc34)cc12] |
| N-[1-(3-chloro-4-fluorophenyl)-2- hydroxyethyl]-4-[4-(3-chlorophenyl)- 1H-pyrazol-3-yl]-1H-pyrrole-2- carboxamide | 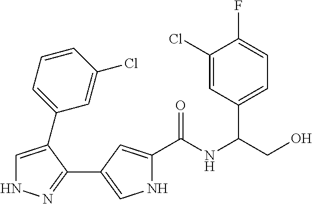 [SMILES: O=C(NC(CO)c1ccc(F)c(Cl)c1)c1cc(-c2n[nH]cc2-c2cccc(Cl)c2)c[nH]1] |

In a further embodiment, at least one of a Ras inhibitor, a MEK inhibitor or an ERK (extracellular signal-regulated kinase) inhibitor (also referred to as a “MAPK (mitogen-activated protein kinase) inhibitor”) can be administered to the subject having a seizure disorder in combination with at least one compound that down regulates Group I mGluR signaling.

“Down regulates mGluR signaling,” as used herein, refers to any decrease or any inhibition in a cellular process or a cellular event or intermediate in a cellular event associated with any mechanism whereby metabotropic glutamate receptors (mGluRs) mediate a biological response. For example, down regulation can be the prevention or any decrease in binding of a signal external to a cell (a first messenger), such as a ligand (e.g., glutamate), to a Group I mGluR. Down regulation can be disruption of a cellular process following binding of an external signal (e.g., ligand) to an mGluR, such as the prevention of activation of adenylyl cyclase or phospholipase C (PLC). Down regulation can also be disruption of a cellular processes following binding of an external signal to Group I mGluR, such as the prevention of activation of a G-protein (Gs, Gq), a decrease in a G-protein (Gs, Gq) activation, prevention of activation of second messengers activated by Group I mGluR (e.g., cAMP, IP₃, diacylglycerol (DAG)) or a decrease in the activity of an intracellular effector, such as a cAMP-dependent protein kinase, protein kinase C (PKC) or calcium release.

Down regulation of Group I mGluR signaling can also be a decrease or inhibition in the release of glutamate. A decrease or inhibition of glutamate release can be through activation of presynaptic Group II and/or Group III mGluRs by, for example, agonists of Group II and Group III mGluRs. An “agonist,” as used herein, is a compound that activates cell signaling. Exemplary Group II and Group III mGluR agonists for use in the invention include LY354740, L-AP4 (Capogna, M. Eur. J. Neurosci. 19: 2847-2858 (2004)) (2R,4R)-4-aminopyrrolidine-2,4-dicarboxylate ((2R,4R)-APDC), (2S,1′S,2′S)-2-(carboxycyclopropyl)glycine (L-CCG-1), N-Acetyl-L-aspartyl-L-glutamic acid (Spaglumic acid), (S)-3-carboxy-4-hydroxyphenylglycine ((S)-3C4HPG), (S)-4-carboxy-3-hydroxyphenylglycine ((S)-4C3HPG) and AMN 082 dihydrochloride (Flor et al., Neuropharmacology 49:244 (2005), the teachings of which are hereby incorporated by reference in its entirety).

Group I mGluR signaling also can be down regulated by decreasing or inhibiting protein synthesis in response to mGluR activation with, for example, rapamycin (Bierer et al., PNAS 87:9231 (1990)). The protein synthesis in response to mGluR activation can be decreased or inhibited by decreasing or inhibiting the transcription, translation, posttranslational modifications, intracellular half-life and/or intracellular processing of signals mediated or activated by mGluR.

In a specific embodiment, suitable compounds that down regulate Group I mGluR can be Group I mGluR antagonists. An mGluR antagonist is a substance which diminishes or abolishes the effect of a ligand (or agonist) that activates an mGluR, for example GluR5 antagonists and mGluR1 antagonists. Exemplary suitable antagonists of mGluR5 are described in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, U.S. Pat. No. 5,672,592, U.S. Pat. No. 5,795,877, U.S. Pat. No. 5,863,536, U.S. Pat. No. 5,880,112 and U.S. Pat. No. 5,902,817, all of which are incorporated by reference in their entirety.

In an embodiment, the subject treated by the methods of the invention has autism. The subject who has autism can further have fragile X syndrome.

In another embodiment, the subject treated by the methods of the invention can have fragile X syndrome. Fragile X mental retardation protein is a protein encoded by the fragile X mental retardation gene 1 (FMR-1). Fragile X syndrome, the most common form of genetically inherited mental retardation, is associated with a fragile site expressed as an isochromatid gap in the metaphase chromosome at map position Xq 27.3. A mutation in the 5′-untranslated region of the FMR1 gene, located on the X chromosome, results in fragile X syndrome (FXS). FMRP is not produced in fragile X syndrome. Genetic testing is available to provide a definitive diagnosis of fragile X syndrome by detecting the mutation in the FMR1 gene and its protein product, FMRP. Genetic testing can be performed on infants, children, adolescents and adults. One of ordinary skill in the art would be able to determine whether a subject has fragile X syndrome by employing established clinical criteria and genetic testing.

In a further embodiment, the subject treated by the methods of the invention can have autism, also referred to herein as “autism spectrum disorder.”

Autism spectrum disorder is a developmental disorder that affects an individual's ability to communicate, form relationships with others and respond appropriately to the environment. Some individuals with autism spectrum disorder are high functioning, with speech and intelligence within normal range. Other individuals with autism spectrum disorder may be nonverbal and/or have varying degrees of mental retardation. Autism spectrum disorder can include Asperger's syndrome and idiopathic autism (e.g., autism of unknown origin). One of ordinary skill in the art would be able to diagnosis an individual with autism spectrum disorder and determine whether the individual has idiopathic autism or Asperger's syndrome, employing well-known clinical criteria as described, for example, in Diagnostic and Statistical Manual of Mental Disorders (DSMMD) (4th ed., pp. 70-71) Washington, D.C., American Psychiatric, 1994.

For example, autism can be diagnosed by assessing the qualitative impairment in social interaction, such as marked impairment in the use of multiple nonverbal behaviors, such as eye-to-eye gaze, facial expression, body postures, gestures to regulate social interaction, failure to develop peer relationships appropriate to developmental level and a lack of spontaneous seeking to share enjoyment, interests, or achievements with other people (e.g., by a lack of showing, bringing, or pointing out objects of interest). The development of spoken language (not accompanied by an attempt to compensate through alternative modes of communication such as gesture or mime), a marked impairment in the ability to initiate or sustain a conversation with others and repetitive use of language or idiosyncratic language. Delayed or abnormal functioning in social interaction, language as used in social communication or symbolic or imaginative play prior to 3 years of age.

In an additional embodiment, the subject that has fragile X syndrome who is treated by the methods of the invention can have autism.

The subject treated by the methods of the invention can be a human subject that is treated as a child, an adolescent and an adult.

In another embodiment, subjects treated by the methods of the invention can have at least one condition selected from the group consisting of Angelman syndrome, Costello syndrome, cardio facio cutaneous syndrome, neurofibromatosis type I, Noonan syndrome and Coffin-Lowry syndrome. One of ordinary skill in the art would be able to diagnosis whether a subject has Angelman syndrome, Costello syndrome, cardio facio cutaneous syndrome, neurofibromatosis type I, Noonan syndrome and Coffin-Lowry syndrome.

Angelman Syndrome is a developmental disorder caused by loss of function of the maternal copy of the UBE3A gene. Symptoms of the disorder include developmental delay, intellectual disability, autism, sleep disturbances, movement disorders, and epilepsy (Mabb, A. M. et al., Trends in Neuroscience, June 2011, volume 34(6), pp. 293-303, the teachings of which are incorporated by reference in their entirety).

Costello Syndrome is a rare disorder caused by mutations in HRAS which result in over-active Ras protein. Symptoms include developmental delay, intellectual disability, loose skin, flexible joints, heart defects, and short stature. Seizures are associated with this disorder (Kawame, H. M. et al., American Journal of Medical Genetics, 2003, volume 118A(1), pp. 8-14; Krab, L. C. et al., Trends in Genetics, 2008, volume 24(10), pp. 498-510; Delrue, M. A. et al, American Journal of Medical Genetics, 2003, volume 123A(3), pp. 301-305, the teachings of all of which are incorporated by reference in their entirety).

Cardio Facio Cutaneous syndrome (CFC) syndrome is a disorder caused by mutations in the BRAF, MAP2K1 or MAP2K2 genes. It is characterized by abnormalities in the heart, skin and facial features, and is associated with developmental delay, intellectual disability, and seizures (Krab, L. C. et al., Trends in Genetics, 2008, volume 24(10), pp. 498-510, the teachings of which are incorporated by reference in their entirety).

Neurofibromatosis Type 1 is a developmental disorder caused by mutations in the NF1 gene. It is characterized by tumors in multiple organ systems and skin discoloration. Learning disabilities and epilepsy are associated with this disorder (Krab, L. C. et al., Trends in Genetics, 2008, volume 24(10), pp. 498-510; Vivarelli, R. S. et al., Journal of Child Neurology, 2003, volume 18(5), pp. 338-342, the teachings of all of which are incorporated by reference in their entirety).

Noonan syndrome is a disorder caused by mutations in the PTPN11, SOS1, RAFT, KRAS, NRAS or BRAF genes. It is characterized by abnormalities in the heart, skeletal structure and circulatory system. Intellectual disability and seizures are associated with this disorder (Krab, L. C. et al., Trends in Genetics, 2008, volume 24(10), pp. 498-510; Adachi, M. et al., Seizure, 2011, epub ahead of print, the teachings of all of which are incorporated by reference in their entirety).

Coffin-Lowry syndrome is a disorder that is caused by mutations in the RPS6KA3 gene. It is characterized by disruptions in multiple organ systems, and is associated with intellectual disability, enhanced acoustic startle response, and seizures (Touraine, R. L. et al., European Journal of Pediatrics, 2002, volume 161(4), pp. 179-187; Krab, L. C. et al., Trends in Genetics, 2008, volume 24(10), pp. 498-510, the teachings of all of which are incorporated by reference in their entirety).

The subject treated by the methods of the invention can have at least one seizure disorder selected from the group consisting of an audiogenic seizure disorder and an epileptic seizure disorder. Seizures, including audiogenic seizures, can occur in individuals with at least one of fragile X syndrome, autism, Angelman syndrome, Costello syndrome, cardio facio cutaneous syndrome, neurofibromatosis type I, Noonan syndrome and Coffin-Lowry syndrome. One of ordinary skill in the art would be able to diagnosis whether a subject has a seizure.

The Ras inhibitor (e.g., farnesyl transferase inhibitor, 3-hydroxy-3-methylglutaryl-Coenzyme A reductase inhibitor) can administered to the subject in a single dose or in multiple doses (i.e., more than one dose). Multiple doses can include multiple daily doses. Multiple doses can be 2, 3, 4, 5, 6, 7, 8, 9, 10 or more doses, which can be administered daily.

The Ras inhibitor (e.g., farnesyl transferase inhibitor, 3-hydroxy-3-methylglutaryl-Coenzyme A reductase inhibitor) can administered to the subject daily, weekly, monthly, yearly or for the lifetime of the subject.

The Ras inhibitor (e.g., farnesyl transferase inhibitor, 3-hydroxy-3-methylglutaryl-Coenzyme A reductase inhibitor), MEK inhibitor, ERK inhibitor, or Group I mGluR antagonist can be administered to the subject in at least one dose selected from the group consisting of a 1 mg dose, 5 mg dose, a 10 mg dose, a 20 mg dose, a 25 mg dose, a 40 mg dose, a 50 mg dose, a 80 mg dose, a 100 mg dose, a 120 mg dose, a 125 mg dose, a 160 mg dose, a 200 mg dose, a 400 mg dose and a 600 mg dose. The dose of the compound administered to the subject can be based on the weight of the subject, for example, a dose such as about 5 mg/kg, about 10 mg/kg, about 20 mg/kg, about 30 mg/kg, about 40 mg/kg, about 50 mg/kg, about 80 mg/kg, about 100 mg/kg, about 150 mg/kg or about 200 mg/kg.

The methods of the invention can be accomplished by the administration of the compounds of the invention (e.g., Ras inhibitors, MEK inhibitor, ERK inhibitor) to a subject (e.g., a human subject) by enteral or parenteral means. The route of administration can be by oral ingestion (e.g., tablet, capsule form) or intramuscular injection of the compound. Other routes of administration can include intravenous, intraarterial, intraperitoneal, or subcutaneous routes, nasal administration, suppositories and transdermal patches. The compounds employed in the methods of the invention can be administered in suitable excipients, including pharmaceutically acceptable salts.

The Ras inhibitors employed in the methods of the invention can include farnesyl transferase inhibitors. Exemplary farnesyl transferase inhibitors can include at least one member selected from the group consisting of a 3-hydroxy-3-methylglutaryl-Coenzyme A reductase inhibitor (e.g., Formulas I-VII), a farnesyl pyrophosphate (FPP) analog (e.g., Formulas VIII-X), a CAAC peptidomimetic (e.g., Formulas XI-XXII), a bisustrate inhibitor (e.g., Formulas XXIII-XXVI) and a nonpeptidomimetic inhibitor (e.g., Formulas XXVII-XXXII). The compounds of Formulas I-XXXII and exemplary doses for use in the methods of the invention are as described below.

LOVASTATIN (Formula I) (1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl-(2S)-2-methylbutanoate; CAS No.: 75330-75-5 (MERCK & CO., INC., Germany) can be administered in a dose range of about 10 to about 100 mg/day.

SIMVASTATIN (Formula II) (1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl 2,2-dimethylbutanoate; CAS No.: 79902-63-9 (MERCK & CO., INC., Germany) can be administered in a dose range of about 5 to about 100 mg/day.

FLUVASTATIN (Formula III) (3R,5S,6E)-7-[3-(4-fluorophenyl)-1-(propan-2-yl)-1H-indol-2-yl]-3,5-dihydropxyhept-6-enoic acid; CAS No.: 93957-54-1 (NOVARTIS) can be administered in a dose range of about 20 to about 100 mg/day.

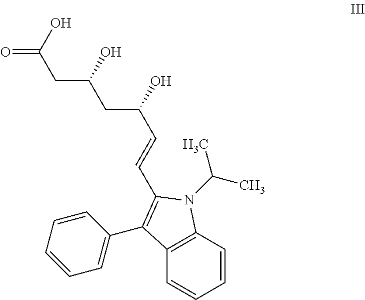 [SMILES: CC(C)n1c(/C=C/[C@@H](O)C[C@@H](O)CC(=O)O)c(-c2ccccc2)c2ccccc21]

PRAVASTATIN (Formula IV) (3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid; CAS No.: 81093-37-0 (BRISTOL-MYERS SQUIBB) can be administered in a dose range of about 20 to about 150 mg/day.

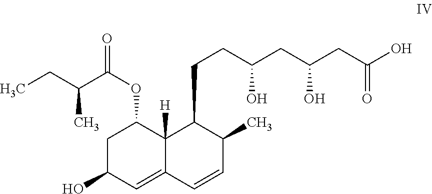 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

ROSUVASTATIN (Formula V) (3R,5S,6E)-7-[4-(4-fluorophenyl)-2-(N-methylmethanesulfonamido)-6-(propan-2-yl)pyrimidin-5-yl]-3,5-dihydroxyhept-6-enoic acid; CAS No.: 287714-41-4 (ASTRAZENECA) can be administered in a dose range of about 5 to about 50 mg/day.

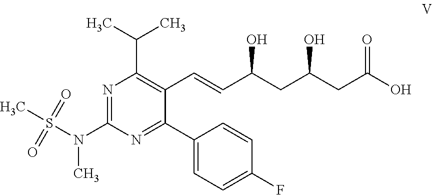 [SMILES: CC(C)c1nc(N(C)S(C)(=O)=O)nc(-c2ccc(F)cc2)c1/C=C/[C@@H](O)C[C@@H](O)CC(=O)O]

PITAVASTATIN (Formula VI) (3R,5S,6E)-7-[2-cyclopropyl-4-(4-fluorophenyl)quinolin-3-yl]-3,5-dihydroxyhept-6-enoic acid; CAS No.: 147511-69-1 (ELI LILLY) can be administered in a dose range of about 1 to about 5 mg/day.

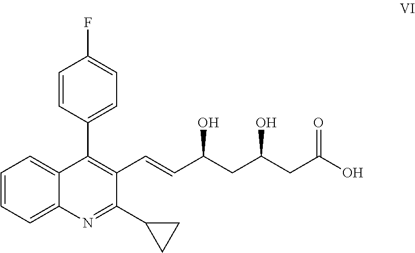 [SMILES: O=C(O)C[C@H](O)C[C@H](O)/C=C/c1c(C2CC2)nc2ccccc2c1-c1ccc(F)cc1]

ATORVASTATIN (Formula VII) (3R,5R)-7-[2-(4-fluorophenyl)-3-phenyl-4-(phenylcarbamoyl)-5-propan-2-ylpyrrol-1-yl]-3,5-dihydroxyheptanoic acid; CAS No.: 134523-00-5 (PFIZER) can be administered in a dose range of about 10 to about 100 mg/day.

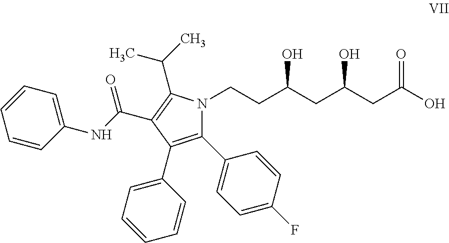 [SMILES: CC(C)c1c(C(=O)Nc2ccccc2)c(-c2ccccc2)c(-c2ccc(F)cc2)n1CC[C@@H](O)C[C@@H](O)CC(=O)O]

Alpha-hydroxyfarnesylphosphonic acid (Formula VIII)

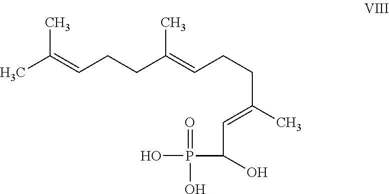 [SMILES: CC(C)=CCC/C(C)=C/CC/C(C)=C/C(O)P(=O)(O)O]

J-104,871 (Formula IX) (4R*,5S*)-5-{N-[(1R,2R,4E)-5-(2-benzoxazolyl)-1-methyl-2-(3,4-methylenedioxyphenyl)-4-pentenyl]-N-(2-naphthylmethyl)carbamoyl}-1,3-dioxolane-2,2,4-tricarboxylicacid can be administered in a dose range of about 1 to about 50 mg/kg/day.

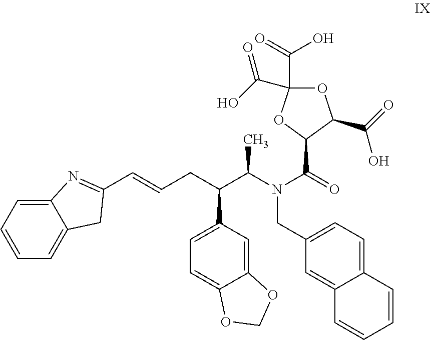 [SMILES: C[C@H]([C@H](C/C=C/C1=Nc2ccccc2C1)c1ccc2c(c1)OCO2)N(Cc1ccc2ccccc2c1)C(=O)[C@H]1OC(C(=O)O)(C(=O)O)O[C@H]1C(=O)O]

RPR-115135 (Formula X) (+/−)-11-[2-(2-methoxyphenyl)acryloyl]-1-phenyl-(1R,8R,9S,13S)-11-azatetracyclo[6.5.2.02,7.09,13]pentadeca-2,4,6-triene-9-carboxylic acid

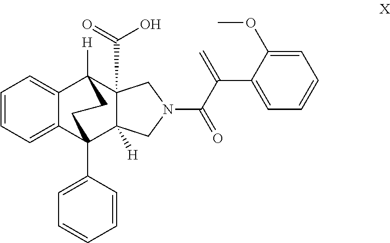

BZA-5B (Formula XI) (Cys-(N-Me)-(BZA)-Met-COOMe); and BZA-2B (Cys-(N-Me)-(BZA)-Met-COOH)

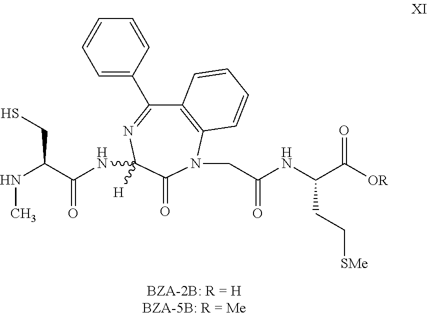

L-731,734 (Formula XII) (2S,3 S)-2-(((2S,3 S)-2-(((R)-2-amino-3-mercaptopropyl)amino)-3-methylpentyl)amino)-3-methyl-N—((S)-2-oxotetrahydrofuran-3-yl)pentanamide

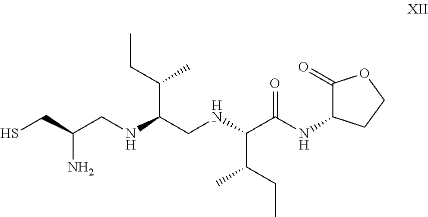 [SMILES: CC[C@H](C)[C@H](NC[C@@H](NC[C@@H](N)CS)[C@@H](C)CC)C(=O)N[C@H]1CCOC1=O]

L-731,735 (Formula XIII) (S)-2-((2S,3S)-2-(((2S,3S)-2-(((R)-2-amino-3-mercaptopropyl)amino)-3-methylpentyl)amino)-3-methylpentanamido)-4-hydroxybutanoic acid

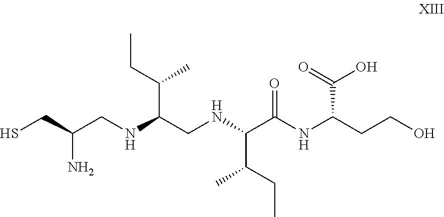 [SMILES: CC[C@H](C)[C@H](NC[C@@H](NC[C@@H](N)CS)[C@@H](C)CC)C(=O)N[C@@H](CCO)C(=O)O]

L-739,749 (Formula XIV) (S)-methyl 2-((S)-2-((2S,3S)-2-(((R)-2-amino-3-mercaptopropyl)amino)-3-methylpentyl)oxy)-3-phenylpropanamido)-4-(methylsulfonyl)butanoate; and L-739,750; (S)-2-((S)-2-(((2S,3S)-2-(((R)-2-amino-3-mercaptopropyl)amino)-3-methylpentyl)oxy)-3-phenylpropanamido)-4-(methylsulfonyl)butanoic acid

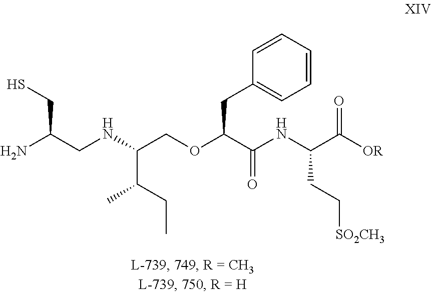

L-739,787 (Formula XV) (S)-2-(((2S,3S)-2-(((R)-2-amino-3-mercaptopropyl)amino)-3-methylpentyl)oxy)-N—((S)-1-hydroxy-4-(methylthio)butan-2-yl)-3-phenylpropanamide

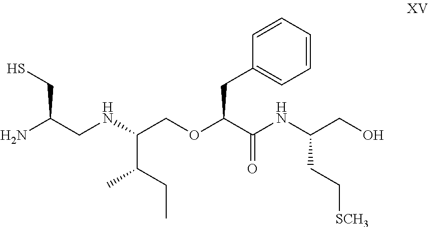 [SMILES: CC[C@H](C)[C@@H](CO[C@@H](Cc1ccccc1)C(=O)N[C@H](CO)CCSC)NC[C@@H](N)CS]

L-744,832 (Formula XVI) (2S)-2-[[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-butanoic acid 1-methylethyl ester

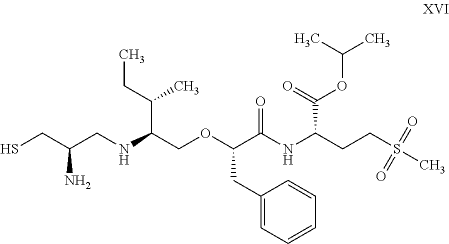 [SMILES: CC[C@H](C)[C@@H](CO[C@@H](Cc1ccccc1)C(=O)N[C@@H](CCS(C)(=O)=O)C(=O)OC(C)C)NC[C@@H](N)CS]

B581 (Formula XVII) (2S)-2-[[(2S)-2-[[(2S)-2-[[(2R)-2-amino-3-sulfanylpropyl]amino]-3-methylbutyl]amino]-3-phenylpropanoyl]amino]-4-methylsulfanylbutanoic acid

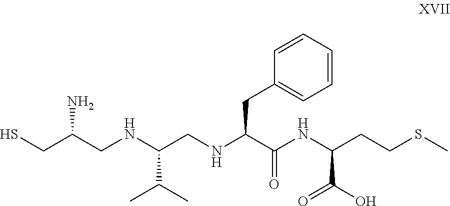 [SMILES: CSCC[C@H](NC(=O)[C@H](Cc1ccccc1)NC[C@@H](NC[C@@H](N)CS)C(C)C)C(=O)O]

Cys-4-aminobenzoic-Met (Formula XVIII)

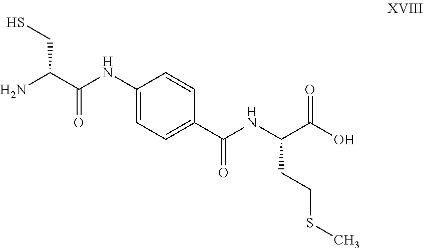 [SMILES: CSCC[C@H](NC(=O)c1ccc(NC(=O)[C@H](N)CS)cc1)C(=O)O]

Cys-aminomethylbenzoic acid-Met (Formula XIX)

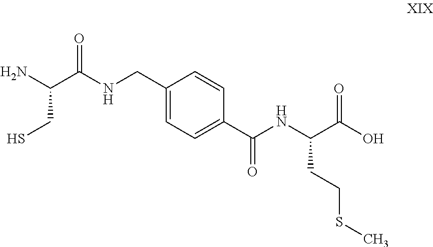 [SMILES: CSCC[C@H](NC(=O)c1ccc(CNC(=O)[C@@H](N)CS)cc1)C(=O)O]

B956 and B1086

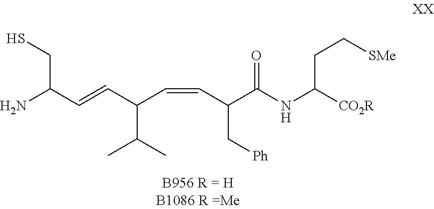

FTI-276 (Formula XXI) (S)-2-(5-(((R)-2-amino-3-mercaptopropyl)amino)-[1,1′-biphenyl]-2-ylcarboxamido)-4-(methylthio)butanoic acid

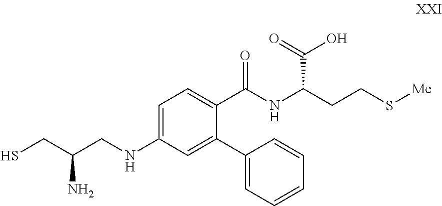 [SMILES: CSCC[C@H](NC(=O)c1ccc(NC[C@@H](N)CS)cc1-c1ccccc1)C(=O)O]

FTI-277 (Formula XXII) (S)-methyl 2-(5-(((R)-2-amino-3-mercaptopropyl)amino)-[1,1′-biphenyl]-2-ylcarboxamido)-4-(methylthio)butanoate

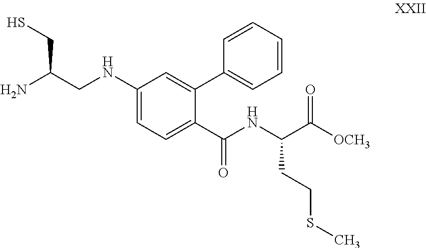 [SMILES: COC(=O)[C@H](CCSC)NC(=O)c1ccc(NC[C@@H](N)CS)cc1-c1ccccc1]

BMS-185878 (Formula XXIII)

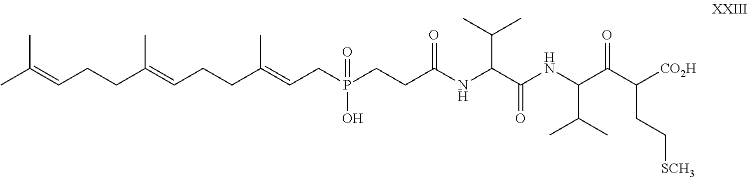 [SMILES: CSCCC(C(=O)O)C(=O)C(NC(=O)C(NC(=O)CCP(=O)(O)C/C=C(\C)CC/C=C(\C)CCC=C(C)C)C(C)C)C(C)C]

BMS-184467 (Formula XXIV)

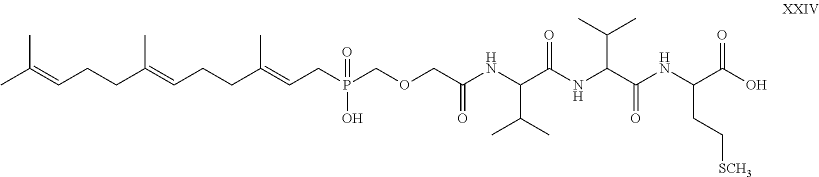 [SMILES: CSCCC(NC(=O)C(NC(=O)C(NC(=O)COCP(=O)(O)C/C=C(\C)CC/C=C(\C)CCC=C(C)C)C(C)C)C(C)C)C(=O)O]

BMS (Formula XXV) 186511 [3-[[(2S)-1-[[(2S)-1-[[(2S)-1-methoxy-4-methylsulfanyl-1-oxobutan-2-yl]amino]-3-methyl-1-oxobutan-2-yl]amino]-3-methyl-1-oxobutan-2-yl]amino]-3-oxopropyl]-[(2E,6E)-3,7,11-trimethyldodeca-2,6,10-trienyl]phosphinic acid

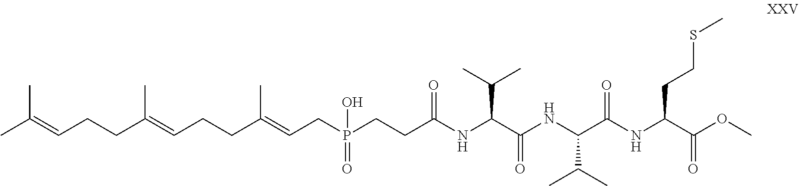 [SMILES: COC(=O)[C@H](CCSC)NC(=O)[C@@H](NC(=O)[C@@H](NC(=O)CCP(=O)(O)C/C=C(\C)CC/C=C(\C)CCC=C(C)C)C(C)C)C(C)C]

BMS-214662 (Formula XXVI) (3R)-3-benzyl-1-(1H-imidazol-5-ylmethyl)-4-thiophen-2-ylsulfonyl-3,5-dihydro-2H-1,4-benzodiazepine-7-carbonitrile can be administered in a dose range about 50 to about 200 mg/kg/week.

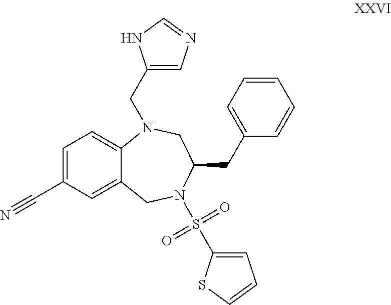 [SMILES: N#Cc1ccc2c(c1)CN(S(=O)(=O)c1cccs1)[C@H](Cc1ccccc1)CN2Cc1cnc[nH]1]

SCH54429 (Formula XXVII) 1-(8-chloro-6,11-dihydro-5H-benzo[5,6]cyclohepta[1,2-b]pyridin-11-yl)-4-(4-pyridinylacetyl)piperazine can be administered in a dose range of about 1 to about 50 mg/kg/day.

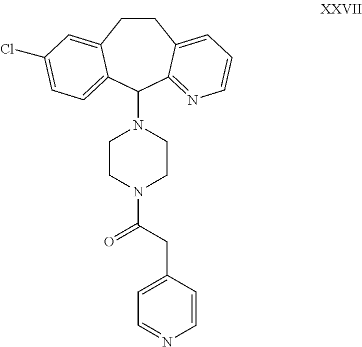 [SMILES: O=C(Cc1ccncc1)N1CCN(C2c3ccc(Cl)cc3CCc3cccnc32)CC1]

LONAFARNIB (Formula)(XVIII) (SCH66336) 4-(2-(4-(8-chloro-3,10-dibromo-6,11-dihydro-5H-benzo(5,6)cyclohepta(1,2-b)pyridin-11-yl)-1-piperidinyl)-2-oxoethyl)-1-piperidinecarboxamide can be administered in a dose range of about 25 to about 125 mg twice/day.

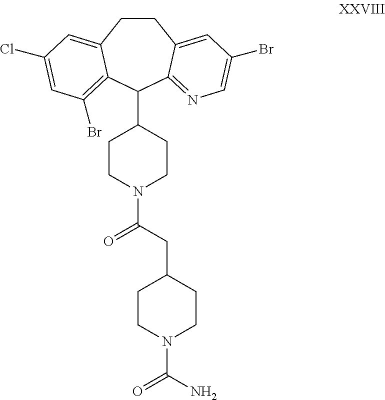 [SMILES: NC(=O)N1CCC(CC(=O)N2CCC(C3c4ncc(Br)cc4CCc4cc(Cl)cc(Br)c43)CC2)CC1]

SCH 44342 (Formula XXIX) 1-(4-pyridylacetyl)-4-(8-chloro-5,6-dihydro-11Hbenzo[5,6] cyclohepta[1,2-b]pyridin-11-ylidene)piperidine can be administered in a dose range of about 1 to about 50 mg/kg/day.

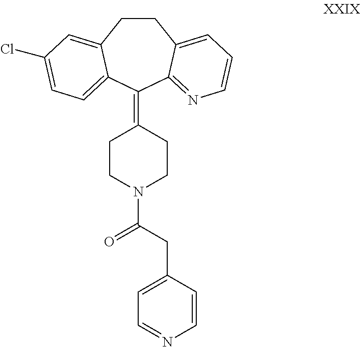 [SMILES: O=C(Cc1ccncc1)N1CCC(=C2c3ccc(Cl)cc3CCc3cccnc32)CC1]

SALIRASIB (Formula XXX) 2-[(2E,6E)-3,7,11-trimethyldodeca-2,6,10-trienyl]sulfanylbenzoic acid can be administered in a dose range about 1 to about 50 mg/kg/day.

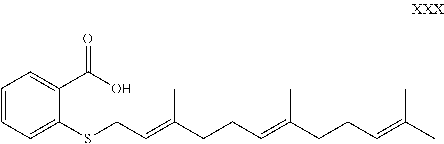 [SMILES: CC(C)=CCC/C(C)=C/CC/C(C)=C/CSc1ccccc1C(=O)O]

ABT-100 (Formula XXXI) 4-[(2S)-2-(4-cyanophenyl)-2-hydroxy-2-(3-methylimidazol-4-yl)ethoxy]-3-[4-trifluoromethoxy)phenyl]benzonitrile can be administered in a dose range of about 1 to about 50 mg/kg/day.

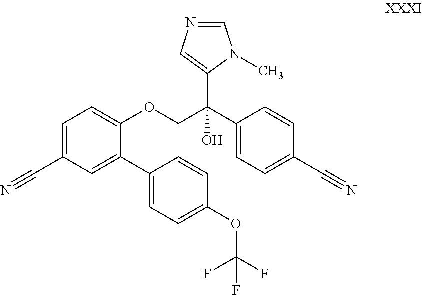 [SMILES: Cn1cncc1[C@](O)(COc1ccc(C#N)cc1-c1ccc(OC(F)(F)F)cc1)c1ccc(C#N)cc1]

TIPIFARNIB (Formula XXXII) (R115777) 6-[(R)-amino-(4-chlorophenyl)-(3-methylimidazol-4-yl)methyl]-4-(3-chlorophenyl)-1-methylquinolin-2-one can be administered in a dose range of about 50 to about 600 mg/day.

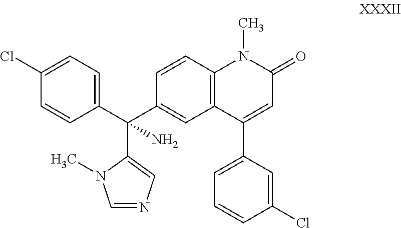 [SMILES: Cn1cncc1[C@@](N)(c1ccc(Cl)cc1)c1ccc2c(c1)c(-c1cccc(Cl)c1)cc(=O)n2C]

### EXEMPLIFICATION

### Example 1

### Reduction in Excessive Protein Synthesis and Audiogenic Seizures

The prevalence of autism spectrum disorders (ASDs) has risen sharply over the past decade [1]. ASDs are developmental disorders characterized by deficits in social interaction, language, communication, and behavior [2]. Fragile X syndrome (FXS) is the leading single-gene cause of autism [3], and the most common form of heritable mental retardation in humans [4]. Characterized by cognitive deficits, developmental delay, and behavioral abnormalities, FXS is caused by the loss of FMRP, the product of the FMR1 gene [5, 6]. Evidence from both in vitro and in vivo studies suggests that FMRP is a repressor of translation [7-10].

Group 1 (Gp1) metabotropic glutamate receptors (mGluR1 and mGluR5) stimulate protein synthesis at synapses [11], and some lasting consequence of mGluR5 activation, such as long-term depression (LTD) of synaptic transmission in the CA1 region of hippocampus, are protein synthesis dependent [12-14]. Based on electrophysiological studies in Fmr1 KO mice, it has been suggested that FMRP represses the synthesis of proteins required for mGluR-LTD [15-17]. These and related data led to the “mGluR theory of FXS”, which proposes that mGluR5 and FMRP act in functional opposition, with mGluR5 driving translation and FMRP repressing this translation [18]. Excessive Gp1 mGluR-mediated protein synthesis in the absence of FMRP is posited to cause the synaptic pathophysiology that gives rise to multiple symptoms of the disease [19].

The activation of Ras is dependent on its interaction with the plasma membrane, which requires posttranslational farnesylation [27-29]. Farnesyl transferase inhibitors (FTIs) prevent the farnesylation of Ras, and thereby reduce its activity. HMG-CoA Reductase inhibitors, such as Formula I, have FTI activity. Formula I crosses the blood brain barrier, and can interfere with p21 Ras farnesylation in the brain [31, 32]. Formula I is well tolerated in humans, and is approved to treat hyperlipidemia in both adults and children [33].

Although some pharmacological interventions exist for the anxiety, epilepsy, and mood disorders associated with fragile X syndrome, there are currently few effective treatment strategies targeted at the core pathophysiological mechanisms underlying seizure disorders in fragile X syndrome and autism [34]. As described herein, an in vitro assay that models the core pathological phenotype of excessive protein synthesis in the Fmr1 KO mouse was employed.

Excessive Protein Synthesis

Materials and Methods

Dorsal hippocampal slices were prepared from juvenile (postnatal day 25-32) WT and Fmr1 KO mice using the same metabolic labeling procedure described in [35]. Briefly, 500 μm slices were recovered in carbogenating, 32.5° C. ACSF (in mM: NaCl: 124, KCl: 3, NaH₂PO₄: 1.25, NaHCO₃: 26, dextrose: 10, MgCl₂: 1, CaCl₂: 2, saturated with 95% O₂ and 5% CO₂), pre-incubated in actinomycin D (ActD, 25 μM) for 30 min to prevent transcription, and protein synthesis measured over 30 minutes via incorporation of a ³⁵S-labeled methionine/cysteine mix (³⁵S-Met/Cys).

Group I mGluR activate several signal pathways. To determine whether a MEK1/2-ERK1/2 inhibitor could rescue excessive protein synthesis in the Fmr1 KO, hippocampal slices were incubated with ±5 μM U0126 (1,4-diamino-2,3-dicyano-1,4-bis[2-aminophenylthio]butadiene) (TOCRIS BIOSCIENCE, Ellisville, Mo.) for 30 minutes, and performed metabolic labeling (±5 μM U0126) for an additional 30 min.

Formula I (LOVASTATIN in lactone form; SIGMA) was evaluated to determine whether it can selectively reduce the excessive protein synthesis seen in the Fmr1 KO. Hippocampal slices were incubated with vehicle or 100 μM Formula I (in lactone form) for 30 minutes, then protein synthesis measured ±100 μM Formula I for an additional 30 minutes.

Results

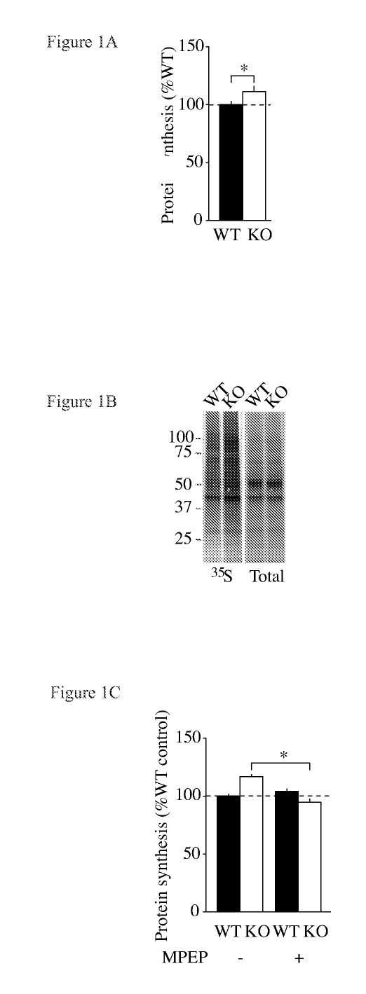

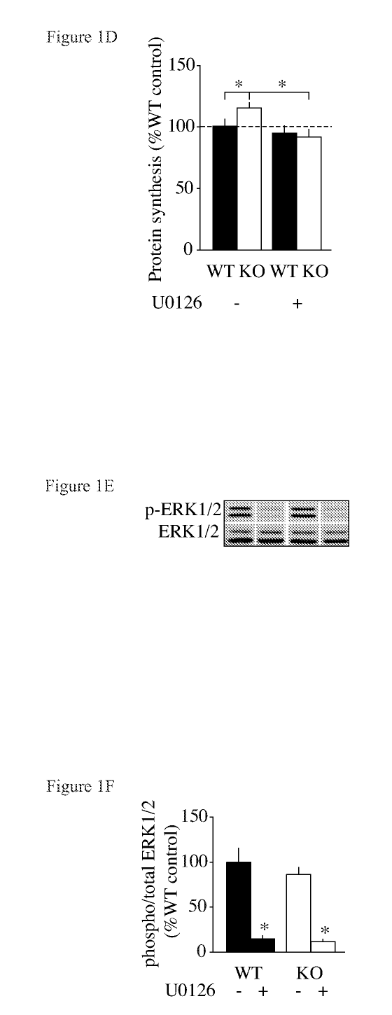

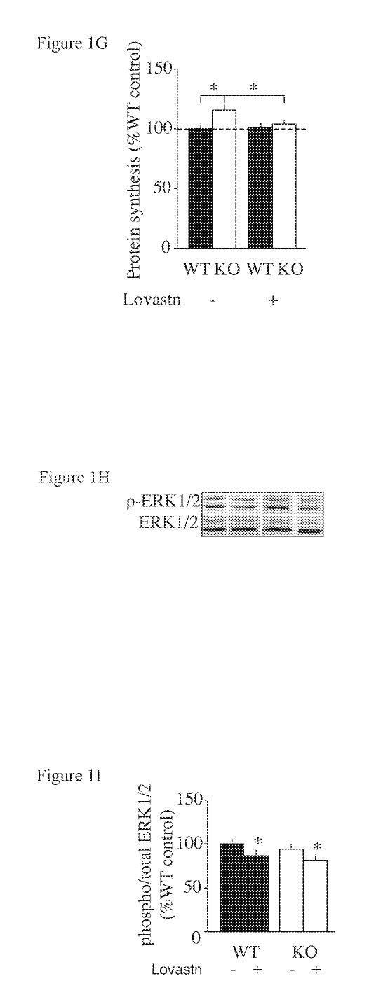

The data described herein show a significant elevation of basal protein synthesis in juvenile Fmr1 KO hippocampal slices as compared to wild type (WT) controls (WT 100±2%, KO 111±4%; n=12; *p=0.0267) (FIGS. 1A and 1B), consistent with previous work in adult Fmr1 KO mice [35, 36].

After 5 minutes of treatment with 50 μM MPEP, an mGluR5 antagonist, protein synthesis in Fmr1 KO hippocampal slices were reduced to WT levels (WT 100±1%, KO 117±2%, WT MPEP 104±2%, KO MPEP 95±2%; n=8; ANOVA genotype x treatment p<0.05; t-test *p=0.0248) (FIG. 1C).

U0126 significantly reduces protein synthesis in Fmr1 KO, but not WT slices (WT: vehicle 100±6%, U0126 94±6%; KO: vehicle 115±4%, U0126 91±6%; n=9; ANOVA treatment p<0.0009, genotype x treatment p<0.03; t-test: KO *p=0.0051, WT p=0.153) (FIGS. 1D and 1E). U0126 treatment significantly decreased ERK1/2 activation (WT: vehicle 100±15%, U0126 15±3%; KO: vehicle 86±8%, U0126 12±2%; n=4; ANOVA treatment p<0.0001, genotype x treatment p=0.606; t-test: WT *p<0.01, KO *p<0.005) (FIGS. 1D and 1E). These data show that downregulation of the ERK1/2 pathway, for example, by compounds that inhibit ERK1/2 activation directly, or indirectly, by, for example, inhibiting activation of MEK signaling, is effective in correcting Fmr1 KO phenotypes, which may be therapeutic in the treatment of seizure disorders of fragile X syndrome and autism.

These data show that Formula I, a Ras inhibitor, results in a significant reduction in ERK1/2 activation (WT: vehicle 100±5%, Formula 187±6%; KO: vehicle 94±5%, Formula 181±6%; n=13; ANOVA genotype p=0.3900, treatment p=0.0037, genotype x treatment p=0.9266; t-test: WT *p=0.0456, KO *p=0.0382), normalizes protein synthesis in the Fmr1 KO down to WT levels (WT: vehicle 100±3%, Formula 1100±3%; KO: vehicle 116±4%, Formula I (LOVASTN)104±2%; n=14; ANOVA genotype p=0.0121, treatment p=0.0660, genotype x treatment p=0.0332; t-test: KO *p=0.0129, WT p=0.815; n=14) (FIGS. 1F and 1G).

Formula I corrects Audiogenic Seizures in the Fmr1 KO

The ability of SL 327 (alpha-[amino[(4-aminophenyl)thio]methylene]-2-(trifluoromethyl)benzeneacetonitrile) (TOCRIS BIOSCIENCE, Ellisville, Mo.), a brain-penetrant MEK1/2-ERK1/2 inhibitor, to correct AGS in the Fmr1 KO mouse. The structural formula of SL 327 is depicted as follows:

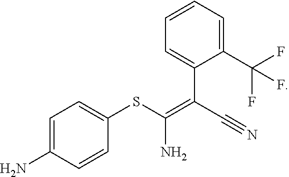 [SMILES: N#C/C(=C(\N)Sc1ccc(N)cc1)c1ccccc1C(F)(F)F]

Materials and Methods

Fmr1 KO and WT mice were injected intraperitoneally (i.p.) with 100 mg/kg SL 327 or vehicle (50% DMSO in ddH2O). One hour later, mice were exposed to a seizure-inducing stimulus for 2 minutes, and scored for four stages of AGS: wild running (WR; pronounced, undirected running and thrashing), clonic seizure (violent spasms accompanied by loss of balance), tonic seizure (postural rigidity in limbs), and death [35, 37, 39]. All animals were injected and scored blind to genotype.

Results

Vehicle-treated Fmr1 KO mice exhibited a 73% incidence of AGS, which was significantly elevated over the 4% incidence observed in vehicle-treated WT mice (Table 3). SL 327 Treatment with SL327 completely eliminated AGS in the Fmr1 KO, dropping the incidence to 0% (Table 3).

To test whether Formula I could also correct AGS in the Fmr1 KO, experiments were repeated using 30 mg/kg Formula I. Treatment with Formula I also corrects AGS in the Fmr1 KO, lowering the incidence to 21% (Table 3). (Fisher's exact test (two-tail): WR: WT control vs. KO control ***p=0.0007, KO control vs. KO SL 327 *p=0.0101, KO control vs. KO Formula I *p=0.0183, KO control vs. WT SL 327 *p=0.0104, KO control vs. WT Formula I **p=0.0076; Clonic WT control vs. KO control *p=0.0106, KO control vs. KO SL 327 *p=0.0462, KO control vs. KO Formula I *p=0.0269, KO control vs. WT SL 327 *p=0.0448, KO control vs. WT Formula I *p=0.0183; Tonic WT control vs. KO control *p=0.0195, KO control vs. KO SL 327 *p=0.0478, KO control vs. KO Formula I *p=0.0125).

TABLE 3
Formula I corrects AGS in the Fmr1 KO

| | WR | Clonic | Tonic | Death | Animals |
|---|---|---|---|---|---|
| KO Control | 73% | 50% | 42% | 19% | 26 |
| KO SL 327 | 0%* | 0%* | 0%* | 0% | 11 |
| KO Formula I | 21%* | 12%* | 6%* | 8% | 33 |
| WT Control | 4%*** | 4%* | 4%* | 0% | 23 |
| WT SL 327 | 0%* | 0%* | 0% | 0% | 10 |
| WT Formula I | 13%** | 8%* | 8% | 8% | 24 |

Fisher's exact test (two-tail), compared to KO Control
*p < 0.05,
**p < 0.01,
***p < 0.001

A significant challenge in the treatment of developmental seizure disorders, particularly in individuals with autism and fragile X syndrome, is finding a pharmaceutical agent that is safe for use in children. The data described herein show that treatment with the Ras inhibitor of Formula I after in vivo administration can decrease and prevent audiogenic seizures, which is a pathological condition observed in fragile X syndrome and autism.

### REFERENCES FOR EXAMPLE 1

1. Kogan, M. D., S. J. Blumberg, L. A. Schieve, C. A. Boyle, J. M. Perrin, R. M. Ghandour, G. K. Singh, B. B. Strickland, E. Trevathan, and P. C. van Dyck, Prevalence of parent-reported diagnosis of autism spectrum disorder among children in the US, 2007. Pediatrics, 2009. 124(5): p. 1395-403.
2. Muhle, R., S. V. Trentacoste, and I. Rapin, The genetics of autism. Pediatrics, 2004. 113(5): p. e472-86.
3. Kelleher, R. J., 3rd and M. F. Bear, The autistic neuron: troubled translation?Cell, 2008. 135(3): p. 401-6.
4. Turner, G., T. Webb, S. Wake, and H. Robinson, Prevalence of fragile X syndrome. Am J Med Genet, 1996. 64(1): p. 196-7.
5. Verkerk, A. J., M. Pieretti, J. S. Sutcliffe, Y. H. Fu, D. P. Kuhl, A. Pizzuti, O. Reiner, S. Richards, M. F. Victoria, and F. P. Zhang, Identification of a gene (FMR-1) containing a CGG repeat coincident with a breakpoint cluster region exhibiting length variation in fragile X syndrome. Cell, 1991. 65(5): p. 905-14.
6. O'Donnell, W. T. and S. T. Warren, A decade of molecular studies of fragile X syndrome. Annu Rev Neurosci, 2002. 25: p. 315-38.
7. Li, Z., Y. Zhang, L. Ku, K. D. Wilkinson, S. T. Warren, and Y. Feng, The fragile X mental retardation protein inhibits translation via interacting with mRNA. Nucleic Acids Res, 2001. 29(11): p. 2276-83.
8. Laggerbauer, B., D. Ostareck, E. M. Keidel, A. Ostareck-Lederer, and U. Fischer, Evidence that fragile X mental retardation protein is a negative regulator of translation. Hum Mol Genet, 2001. 10(4): p. 329-38.
9. Mazroui, R., M. E. Huot, S. Tremblay, C. Filion, Y. Labelle, and E. W. Khandjian, Trapping of messenger RNA by Fragile X Mental Retardation protein into cytoplasmic granules induces translation repression. Hum Mol Genet, 2002. 11(24): p. 3007-17.
10. Aschrafi, A., B. A. Cunningham, G. M. Edelman, and P. W. Vanderklish, The fragile X mental retardation protein and group I metabotropic glutamate receptors regulate levels of mRNA granules in brain. Proc Natl Acad Sci USA, 2005. 102(6): p. 2180-5.
11. Weiler, I. J., S. A. Irwin, A. Y. Klintsova, C. M. Spencer, A. D. Brazelton, K. Miyashiro, T. A. Comery, B. Patel, J. Eberwine, and W. T. Greenough, Fragile X mental retardation protein is translated near synapses in response to neurotransmitter activation. Proc Natl Acad Sci USA, 1997. 94(10): p. 5395-400.
12. Huber, K. M., M. S. Kayser, and M. F. Bear, Role for rapid dendritic protein synthesis in hippocampal mGluR-dependent long-term depression. Science, 2000. 288(5469): p. 1254-7.
13. Merlin, L. R., P. J. Bergold, and R. K. Wong, Requirement of protein synthesis for group I mGluR-mediated induction of epileptiform discharges. J Neurophysiol, 1998. 80(2): p. 989-93.
14. Vanderklish, P. W. and G. M. Edelman, Dendritic spines elongate after stimulation of group 1 metabotropic glutamate receptors in cultured hippocampal neurons. Proc Natl Acad Sci USA, 2002. 99(3): p. 1639-44.
15. Nosyreva, E. D. and K. M. Huber, Metabotropic receptor-dependent long-term depression persists in the absence of protein synthesis in the mouse model of fragile X syndrome. J Neurophysiol, 2006. 95(5): p. 3291-5.
16. Hou, L., M. D. Antion, D. Hu, C. M. Spencer, R. Paylor, and E. Klann, Dynamic translational and proteasomal regulation of fragile X mental retardation protein controls mGluR-dependent long-term depression. Neuron, 2006. 51(4): p. 441-54.
17. Huber, K. M., S. M. Gallagher, S. T. Warren, and M. F. Bear, Altered synaptic plasticity in a mouse model of fragile X mental retardation. Proc Natl Acad Sci USA, 2002. 99(11): p. 7746-50.
18. Bear, M. F., K. M. Huber, and S. T. Warren, The mGluR theory of fragile X mental retardation. Trends Neurosci, 2004. 27(7): p. 370-7.
19. Bear, M. F., Therapeutic implications of the mGluR theory of fragile X mental retardation. Genes Brain Behav, 2005. 4(6): p. 393-8.
20. Ferraguti, F., B. Baldani-Guerra, M. Corsi, S, Nakanishi, and C. Corti, Activation of the extracellular signal-regulated kinase 2 by metabotropic glutamate receptors. Eur J Neurosci, 1999. 11(6): p. 2073-2082.
21. Mao, L., L. Yang, Q. Tang, S. Samdani, G. Zhang, and J. Q. Wang, The scaffold protein Homer 1b/c links metabotropic glutamate receptor 5 to extracellular signal-regulated protein kinase cascades in neurons. J Neurosci, 2005. 25(10): p. 2741-52.
22. Choe, E. S. and J. Q. Wang, Group I metabotropic glutamate receptors control phosphorylation of CREB, Elk-1 and ERK via a CaMKII-dependent pathway in rat striatum. Neurosci Lett, 2001. 313(3): p. 129-32.
23. Adwanikar, H., F. Karim, and R. W.t. Gereau, Inflammation persistently enhances nocifensive behaviors mediated by spinal group I mGluRs through sustained ERK activation. Pain, 2004. 111(1-2): p. 125-35.
24. Berkeley, J. L. and A. I. Levey, Cell-specific extracellular signal-regulated kinase activation by multiple G protein-coupled receptor families in hippocampus. Mol Pharmacol, 2003. 63(1): p. 128-35.
25. Gallagher, S. M., C. A. Daly, M. F. Bear, and K. M. Huber, Extracellular signal-regulated protein kinase activation is required for metabotropic glutamate receptor-dependent long-term depression in hippocampal area CA1. J Neurosci, 2004. 24(20): p. 4859-64.
26. Garcia, S., E. Lopez, and A. M. Lopez-Colome, Glutamate accelerates RPE cell proliferation through ERK1/2 activation via distinct receptor-specific mechanisms. J Cell Biochem, 2008. 104(2): p. 377-90.
27. Elad, G., A. Paz, R. Haklai, D. Marciano, A. Cox, and Y. Kloog, Targeting of K-Ras 4B by S-trans,trans-farnesyl thiosalicylic acid. Biochim Biophys Acta, 1999. 1452(3): p. 228-42.
28. Haklai, R., M. G. Weisz, G. Elad, A. Paz, D. Marciano, Y. Egozi, G. Ben-Baruch, and Y. Kloog, Dislodgment and accelerated degradation of Ras. Biochemistry, 1998. 37(5): p. 1306-14.
29. Rotblat, B., M. Ehrlich, R. Haklai, and Y. Kloog, The Ras inhibitor farnesylthiosalicylic acid (Salirasib) disrupts the spatiotemporal localization of active Ras: a potential treatment for cancer. Methods Enzymol, 2008. 439: p. 467-89.
30. Agrawal, A. G. and R. R. Somani, Farnesyltransferase inhibitor as anticancer agent. Mini Rev Med Chem, 2009. 9(6): p. 638-52.
31. Guilding, C., K. McNair, T. W. Stone, and B. J. Morris, Restored plasticity in a mouse model of neurofibromatosis type 1 via inhibition of hyperactive ERK and CREB. Eur J Neurosci, 2007. 25(1): p. 99-105.
32. Li, W., Y. Cui, S. A. Kushner, R. A. Brown, J. D. Jentsch, P. W. Frankland, T. D. Cannon, and A. J. Silva, The HMG-CoA reductase inhibitor Formula I reverses the learning and attention deficits in a mouse model of neurofibromatosis type I. Curr Biol, 2005. 15(21): p. 1961-7.
33. Corvol, J. C., A. Bouzamondo, M. Sirol, J. S. Hulot, P. Sanchez, and P. Lechat, Differential effects of lipid-lowering therapies on stroke prevention: a meta-analysis of randomized trials. Arch Intern Med, 2003. 163(6): p. 669-76.
34. Hagerman, R. J., E. Berry-Kravis, W. E. Kaufmann, M. Y. Ono, N. Tartaglia, A. Lachiewicz, R. Kronk, C. Delahunty, D. Hessl, J. Visootsak, J. Picker, L. Gane, and M. Tranfaglia, Advances in the treatment of fragile X syndrome. Pediatrics, 2009. 123(1): p. 378-90.
35. Dolen, G., E. Osterweil, B. S. Rao, G. B. Smith, B. D. Auerbach, S. Chattarji, and M. F. Bear, Correction of fragile X syndrome in mice. Neuron, 2007. 56(6): p. 955-62.
36. Qin, M., J. Kang, T. V. Burlin, C. Jiang, and C. B. Smith, Postadolescent changes in regional cerebral protein synthesis: an in vivo study in the FMR1 null mouse. Neurosci, 2005. 25(20): p. 5087-95.
37. Yan, Q. J., M. Rammal, M. Tranfaglia, and R. P. Bauchwitz, Suppression of two major Fragile X Syndrome mouse model phenotypes by the mGluR5 antagonist MPEP. Neuropharmacology, 2005. 49(7): p. 1053-66.
38. Berry-Kravis, E., Epilepsy in fragile X syndrome. Dev Med Child Neurol, 2002. 44(11): p. 724-8.
39. Zhong, J., S. C. Chuang, R. Bianchi, W. Zhao, H. Lee, A. A. Fenton, R. K. Wong, and H. Tiedge, BC1 regulation of metabotropic glutamate receptor-mediated neuronal excitability. J Neurosci, 2009. 29(32): p. 9977-86.

### Example 2

### Reduction in Excessive Protein Synthesis and Audiogenic Seizures

Epilepsy is one of the most debilitating symptoms associated with autism spectrum disorders (ASD). The onset of epilepsy (epileptogenesis) within the first few years of life is correlated with increased risk of developing ASD[1]. Despite this, there remains little understanding of the mechanisms linking epilepsy to autism, and no targeted treatments for preventing epileptogenesis[2]. The most prevalent single-gene cause of autism is fragile X syndrome (FXS), a developmental disorder associated with intellectual disability and a high incidence of epilepsy[3]. The pathogenesis of FXS has been linked to excessive cerebral protein synthesis, which is downstream of constitutive activation of group I metabotropic glutamate receptor 5 (mGluR5)[4]. As described herein, Formula I (LOVASTATIN), a drug with an excellent safety profile that is prescribed and approved to treat hypercholesterolemia in children[9], decreases ERK1/2 signaling, normalizes excessive protein synthesis, and corrects AGS in the Fmr1 KO. LOVASTATIN, a Ras inhibitor, may be useful in the methods described herein as a treatment to block epileptogenesis in subjects, including humans with autism and humans with autism and fragile X syndrome.

Both autism and epilepsy are increasingly prevalent conditions, each affecting an estimated 1 in 100 individuals[10, 11]. About 30% of the ASD population has epilepsy. Studies have found that the manifestation of seizures within the first 3 years of life correlate with an increased incidence of ASD in at-risk children[2]. Childhood epileptogenesis may contribute to the progression of autism, underscoring the need for an effective prophylaxis for epilepsy that is safe for use during development. The genetic heterogeneity of autism, and of epilepsy, has made it difficult understand the molecular mechanisms linking the two disorders. To mitigate this complexity, a mouse model of FXS, a single-gene developmental disorder associated with about 30% incidence of both autism and epilepsy[13, 14] was employed in these studies. The Fmr1 KO is a suitable model because it involves the loss of a single gene of known function, and thus the genetic contribution to the pathophysiology can be attributed to loss of FMRP function. In addition, robust epilepsy phenotypes occur both in vitro, as measured by ictal discharges in hippocampal slices, and in vivo, as measured by increased susceptibility to AGS[14, 15] in the fragile X knock out mouse.

The FMR1 gene encodes for the translational repressor Fragile X Mental Retardation Protein (FMRP). Several pathological changes observed in FXS may stem from an elevation of basal protein synthesis in the nervous system due to a loss of FMRP. mGluR5-ERK1/2 pathway may be a contributor to the pathogenesis of FXS[16] Inhibition of mGluR5 or ERK1/2, as described herein, corrects the AGS phenotype observed in the Fmr1 KO mouseERK1/2 is a member of the larger MAP kinase (MAPK) signaling pathway, headed by the small GTPase Ras[17]. The HMG-CoA reductase inhibitor of Formula I has been shown to reduce ERK1/2 signaling in neurons in vitro and in vivo by limiting Ras activation. The maturation of Ras to its active conformation requires the addition of a farnesyl group, which facilitates interaction with the plasma membrane, and Formula I negatively regulates this process[20, 21]. Unlike many proposed therapies to treat seizure disorders, Formula I has an advantage because it has been approved by the Food and Drug Administration (FDA) to treat humans, including treatment of hypercholesterolemia in children [9, 22]. Formula I has an excellent safety profile, and is widely prescribed for long-term treatment in both adults and children.

Materials and Methods

Mice: Fmr1 KO (Jackson Labs) and WT littermates, bred on the C57BL6 or FVB background strains, were group housed and maintained in a 12:12 h light:dark cycle. An In vitro hippocampal slice assay was used herein to recapitulate the exaggerated protein synthesis phenotype seen in the Fmr1 KO[6]. Hippocampal slices were prepared from juvenile Fmr1 KO and wild type (WT) mice, pre-incubated in 10-50 μM Formula I or vehicle for 30 minutes, and protein synthesis measured for another 30 min via incorporation of a ³⁵S-labeled methionine/cysteine mix.

Drugs: Formula I acid (EMD Biosciences), Formula I lactone (SIGMA), and actinomycin D (TOCRIS) were reconstituted according to manufacturer's instructions.

Metabolic labeling: Experiments were performed on male WT and Fmr1 KO mice (postnatal day 25-32), blind to genotype, as described herein. Briefly, 500 μm slices were recovered for 4 h in 32.5° C. ACSF (in mM: 124 NaCl, 3 KCl, 1.25 NaH₂PO₄, 26 NaHCO₃, 10 dextrose, 1 MgCl₂, 2 CaCl₂, saturated with 95% O₂ and 5% CO₂), incubated for 30 min with 25 μM ActD±Formula I (10 μM or 50 μM), and transferred to fresh ACSF±drug with 10 μCi/ml ³⁵S-Met/Cys (Perkin Elmer) for another 30 min. After labeling, slices were homogenized, and labeled proteins isolated by TCA precipitation. Samples were read with a scintillation counter and also subjected to a protein concentration assay (BIO-RAD). Final data were expressed as counts per minute (CPM) per μg protein, normalized to the ³⁵S-Met/Cys ACSF used for incubation, and the average incorporation of all samples analyzed in that experiment.

Immunoblotting: Immunoblotting was performed, blind to genotype and treatment as described herein using primary antibodies to Ras (PIERCE), p-ERK1/2 (Thr202/Tyr204), or ERK1/2 (CELL SIGNALING TECHNOLOGY), and HRP-conjugated secondary antibodies (GE HEALTHCARE).

Ras activation assay: Slices (4-6 per animal) were prepared exactly as for metabolic labeling, and Ras-GTP isolated using the Active Ras Pull-Down and Detection kit (PIERCE) according to manufacturer's instructions.

AGS: Experiments were performed using male WT and Fmr1 KO littermates (P17-25) as described herein. For acute exposure experiments, mice were weighed and injected i.p. with: (A) 30 mg/kg Formula I acid or vehicle (10% DMSO in saline), (B) 100 mg/kg Formula I acid or vehicle (33% DMSO in saline), or (C) 30 mg/kg Formula I lactone or vehicle (50% DMSO in saline), returned to their home cages for 1 h. For oral administration experiments, mice were weaned onto standard rodent chow (BIO-SERV) formulated with 100 mg/kg IC Formula I (40 mg tablets, MYLAN INC.) or identical chow containing no Formula I, and allowed to feed ad libitum for 48 hours. Drug doses were chosen on based upon previous work [19, 23, 24]. Immediately prior to testing, mice were transferred to a quiet (about <60 dB ambient sound) room for 1 hour. Mice were then transferred to a transparent plastic test chamber and, after 1 minute of habituation, exposed to a 130 dB stimulus (recorded sampling of a modified personal alarm, RADIOSHACK model 49-1010) for about 2 minutes. Each testing session contained mice from both genotype and treatment groups. For each group, incidence of the following stages of AGS was calculated: wild running (WR; pronounced, undirected running and thRashing), clonic seizure (violent spasms accompanied by loss of balance), tonic seizure (postural rigidity in limbs), and death. All aspects of the experiments were performed blind to genotype.

Statistics: For biochemistry experiments, outliers greater than 2 standard deviations from the mean were removed, and significance was determined by two-way repeated measures mixed model ANOVA. If significant effects were found by ANOVA, post hoc analyses were performed to compare individual groups using two-tailed paired or unpaired (for Ras activation assays) Student's t-tests. For AGS experiments, significance was determined by two-tailed Fisher's exact test (for overall incidence) or Kolmogorov-Smirnov test (for severity score distribution).

### RESULTS AND DISCUSSION

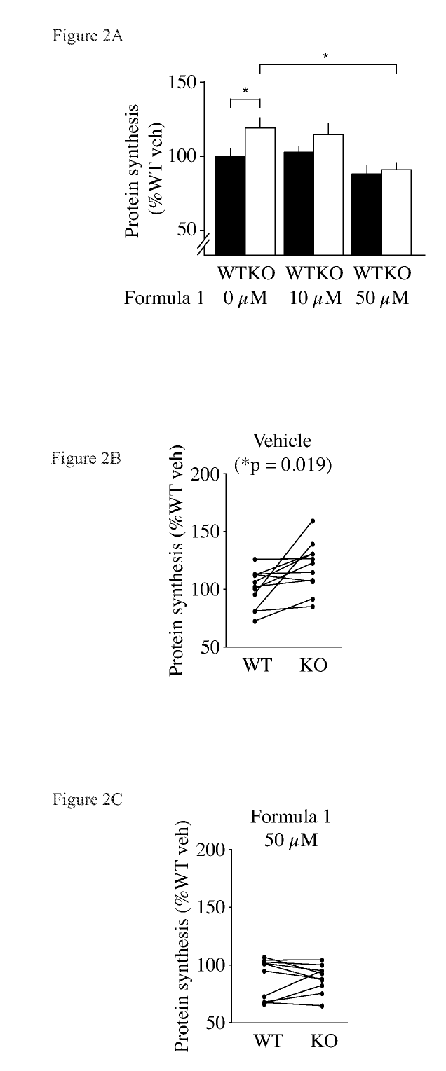

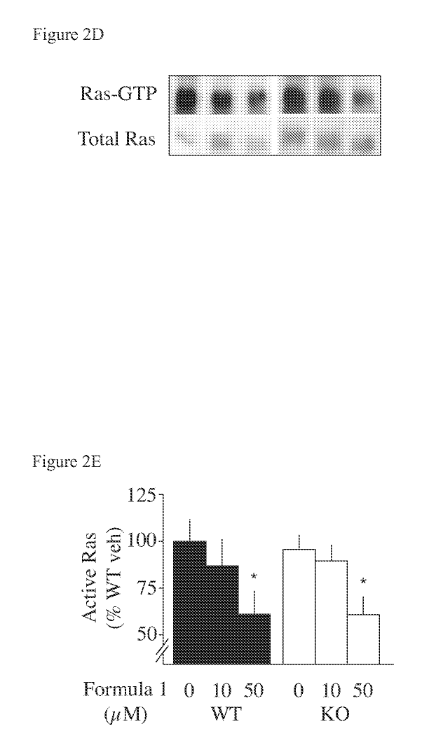

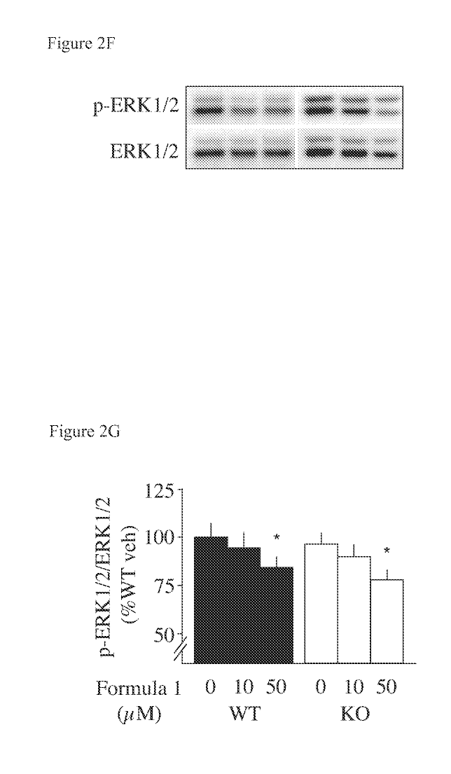

As described herein, 50 μM Formula I normalizes protein synthesis in the Fmr1 KO down to WT levels (WT: veh 100±5%, 10 μM 103±4%; 50 μM 88±5%; KO: veh 119±6%, 10 μM 115±7%; 50 μM 91±4%; n=11; ANOVA genotype *p=0.0121, treatment p=0.0660, genotype x treatment *p=0.0332; post hoc t-test: WT vs. KO veh *p=0.019, KO veh vs. 50 μM *p=0.011) (FIGS. 2A-2C). These results show that Formula I is sufficient to normalize the excessive protein synthesis seen in the Fmr1 KO.

To examine whether Formula I application significantly inhibits Ras-ERK1/2 activation, the amount of active Ras in Formula I-treated slices was measured. Hippocampal slices were prepared from WT and Fmr1 KO, then incubated with 10-50 μM Formula I or vehicle as described above. Active (GTP-bound) Ras was isolated from homogenized slices using a GST-pull down assay, and compared to total Ras. These data show that 50 μM Formula I significantly reduces the amount of active Ras in both WT and Fmr1 KO hippocampal slices (WT: veh 100±11%, 10 μM 87±14%; 50 μM 61±12%; KO: veh 96±8%, 10 μM 89±8%; 50 μM 61±9%; ANOVA genotype x treatment *p=0.0462; n=8; post hoc t-test: WT veh vs. 50 μM *p=0.039, KO veh vs. 50 μM *p=0.015) (FIGS. 2D and 2E).

To determine whether the observed downregulation of active Ras was accompanied by a concomitant decrease in ERK1/2 signaling, ERK1/2 phosphorylation in hippocampal slices exposed to 10-50 μM Formula I or vehicle for 1 hour was determined. Results from Western blot analysis reveal that 50 μM Formula I significantly reduces ERK1/2 activation in both WT and Fmr1 KO slices (WT: veh 100±7%, 10 μM 94±8%; 50 μM 84±5%; KO: veh 97±5%, 10 μM 90±6%; 50 μM 78±5%; ANOVA genotype p=0.3900, treatment *p=0.0037, genotype x treatment p=0.9266; n=13; post hoc t-test: WT veh vs. 50 μM *p=0.035, KO veh vs. 50 μM *p=0.008) (FIGS. 2F and 2G). These data show that Formula I effectively inhibits the Ras-ERK1/2 pathway in hippocampal slices. Excessive protein synthesis in the Fmr1 KO may be due to a hypersensitivity to, not hyperactivation of, the ERK1/2 signaling pathway. The methods described herein inhibit activation of Ras signaling by inhibit Ras.

The in vitro studies indicated that Formula I is effective in correcting the pathological excess in protein synthesis seen in the Fmr1 KO. To determine whether a reduction of epileptogenesis could be observed in vivo, AGS susceptibility in Fmr1 KO mice acutely injected with Formula I was determined. Enhanced susceptibility to AGS is one of the most robust phenotypes observed in the Fmr1 KO mouse, and is used to model the epilepsy observed in FXS patients [14, 15].

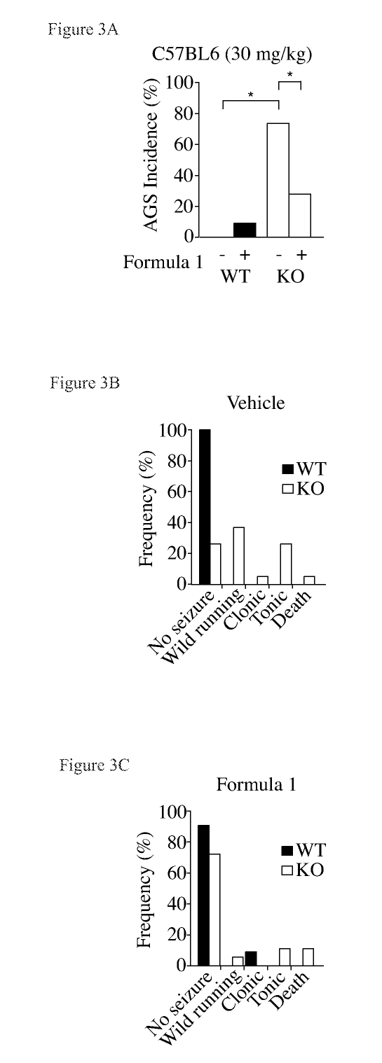

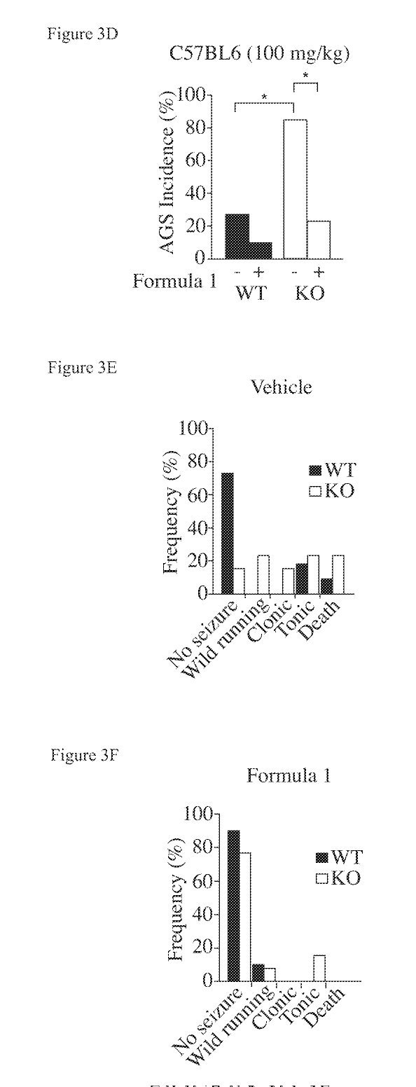

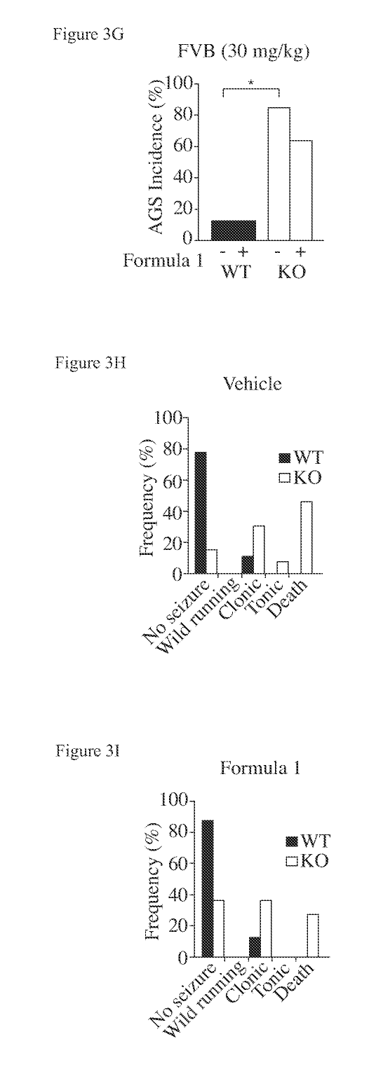

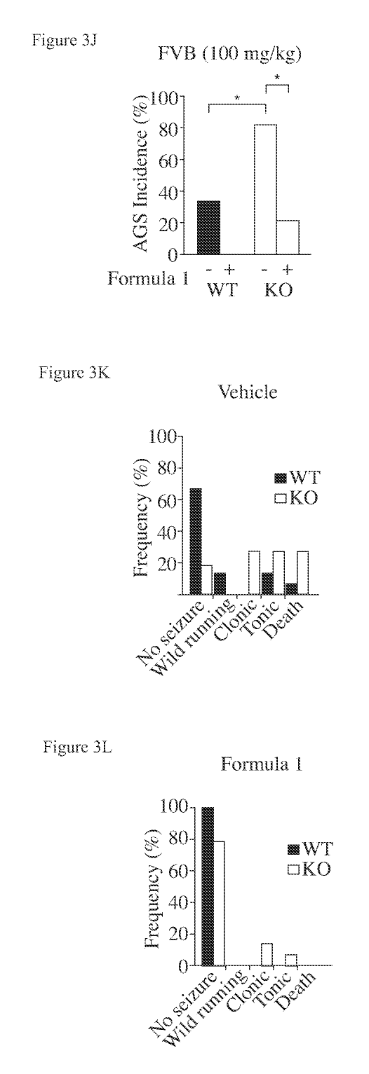

Fmr1 KO and WT mice were injected intraperitoneally (i.p.) with 30 mg/kg Formula I and, after one hour, exposed to a 130 dB seizure-inducing stimulus for 2 minutes (see Materials and Methods). For each mouse, four stages of increasing AGS severity were scored: wild running, clonic seizure, tonic seizure, or death[16]. Vehicle-treated Fmr1 KO mice exhibited about 74% incidence of AGS, and no seizures were observed in vehicle-treated WT mice (Fisher's exact test (FET): WT vs. KO *p=0.0002; FIG. 3A). Acute injection of Formula I reduced the incidence of AGS to about 28% in the Fmr1 KO (FET: KO veh vs. lov *p=0.009; n=18-19) (FIG. 3A). These results show that acute Formula I administration significantly reduces seizure activity in the Fmr1 KO. To assess whether Formula I affected the severity of seizures in the Fmr1 KO, the distribution of AGS scores was compared between vehicle and Formula I treated groups. These data show that the significant difference in AGS severity between WT and Fmr1 KO mice in the vehicle treated group (Kolmogorov-Smirnov test (K-S test): WT vs. KO veh *p=0.002) is eliminated with Formula I treatment (K-S test: WT vs. KO lov p=0.999; KO veh vs. lov *p=0.041) (FIGS. 3B and 3C). These results show that Formula I significantly lessens the severity of seizures observed in the Fmr1 KO. Experiments using a higher dose of 100 mg/kg Formula I revealed a similar reduction in AGS incidence (KO veh vs. lov 85% to 23%; FET *p=0.005; n=13) and severity (K-S test: KO veh vs. lov *p=0.015; WT vs. KO veh. *p=0.04; WT vs. KO lov p=0.999) (FIGS. 3D-3F).

The Fmr1 KO AGS phenotype is seen in multiple mouse strains[15]. The beneficial effects of Formula I on AGS susceptibility in different strains of mice, C57BL6 mice and Fmr1 KO mice bred on the FVB background strain was determined. About 85% incidence of AGS in vehicle treated Fmr1 KO FVB mice[15] was observed, which was significantly higher than the 11% incidence observed in vehicle treated WT mice (FET: *p=0.002). Interestingly, Fmr1 KO FVB mice injected with 30 mg/kg Formula I exhibit about 64% incidence of AGS incidence, which is not significantly different from the vehicle treated group (FET: p=0.357; FIG. 3G). The severity of AGS is similarly unaffected by this treatment (K-S test: KO veh vs. lov p=0.862) (FIGS. 3H and 31). Studies have shown a mouse strain-dependent difference in Formula I metabolism[23]. These experiments were repeated using a higher dose. Results from these experiments show that about 100 mg/kg Formula I significantly lowers AGS incidence (FET: KO veh 82%, KO lov 21%, *p=0.005; n=11-14; FIG. 3J) and reduces AGS severity (K-S test: KO veh vs. lov *p=0.022; WT vs. KO veh. *p=0.016; WT vs. KO lov p=0.999) in FVB Fmr1 KO mice (FIGS. 3K and 3L). These results show that Formula I is effective in correcting AGS in Fmr1 KO mice bred on multiple background strains.

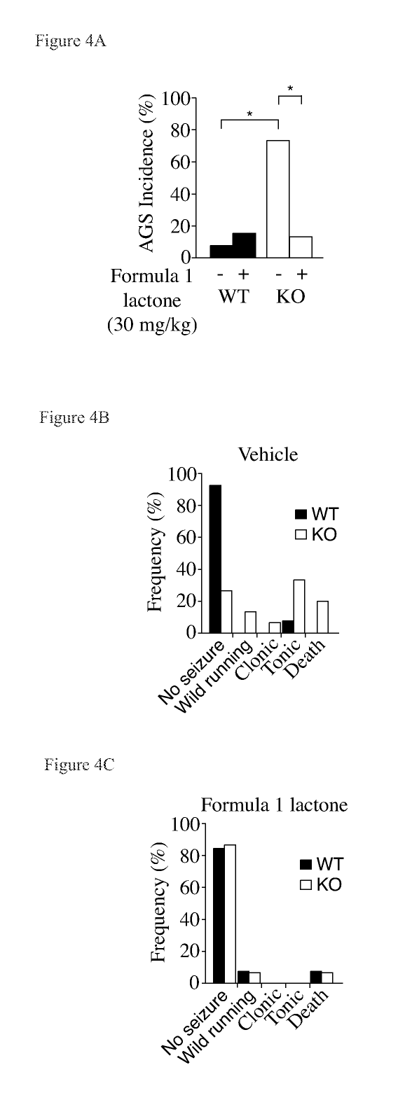

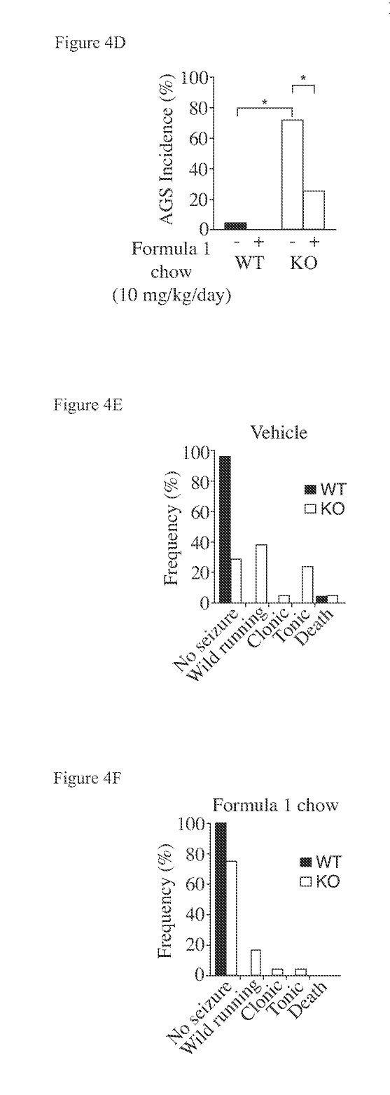

In patients, Formula I is administered in its lactone form, which is metabolized into an active conformation (Formula I acid) in the body[9]. While our in vitro studies required the use of Formula I acid, confirmation that the in vivo administration of Formula I lactone could reduce AGS incidence and severity in the Fmr1 KO was made in AGS experiments using 30 mg/kg Formula I lactone. Formula I lactone significantly reduces AGS incidence from about 73% to about 20% (FET: *p=0.009; n=15; FIG. 4A) and reduces AGS severity (K-S test: KO veh vs. lov *p=0.009; WT vs. KO veh. *p=0.005; WT lov vs. lov p=0.999) in the Fmr1 KO (FIGS. 4B and 4C).

To further investigate the viability of Formula I as a treatment in humans, Formula I was orally administrated to correct AGS incidence and severity in the Fmr1 KO. WT and Fmr1 KO mice were fed standard rodent chow supplemented with about 0.1% Formula I, which corresponds to a dose of about 10 mg/kg/day[24]. After a 48 hour exposure to either Formula I chow or control chow with the same formulation, mice were tested for AGS. Oral administration of Formula I significantly reduces AGS incidence (FET: KO con 71%, KO lov 25%, *p=0.003; n=21-24; FIG. 4D) and severity (K-S test: KO veh vs. lov *p=0.016; WT vs. KO veh. *p=0.0001; WT vs. KO lov p=0.461) in the Fmr1 KO mouse (FIGS. 4E and 4F). These results show that oral administration of pharmaceutical grade Formula I is effective in correcting AGS susceptibility in the Fmr1 KO.

Preventing epileptogenesis in ASD patients could have a dramatic impact on the progression and severity of humans with epilepsy, including humans with autism and humans with fragile X syndrome who are autistic. Given the increasing prevalence of ASD, identifying an effective strategy for preventing epileptogenesis is of particular urgency. Ras inhibitors, such as Formulas I-XXXII may correct excessive protein synthesis and significantly reduces the incidence and severity of AGS in the Fmr1 KO. Ras inhibitors may be useful to treat seizures in FXS and other autism-linked developmental disorders.

### REFERENCES FOR EXAMPLE 2

1. Tuchman, R., M. Cuccaro, and M. Alessandri, Autism and epilepsy: historical perspective. Brain Dev, 2010. 32(9): p. 709-18.
2. Tuchman, R., M. Alessandri, and M. Cuccaro, Autism spectrum disorders and epilepsy: moving towards a comprehensive approach to treatment. Brain Dev, 2010. 32(9): p. 719-30.
3. Kelleher, R. J., 3rd and M. F. Bear, The autistic neuron: troubled translation?Cell, 2008. 135(3): p. 401-6.
4. Qin, M., J. Kang, T. V. Burlin, C. Jiang, and C. B. Smith, Postadolescent changes in regional cerebral protein synthesis: an in vivo study in the FMR1 null mouse. J Neurosci, 2005. 25(20): p. 5087-95.
5. O'Donnell, W. T. and S. T. Warren, A decade of molecular studies of fragile X syndrome. Annu Rev Neurosci, 2002. 25: p. 315-38.
6. Dolen, G., E. Osterweil, B. S. Rao, G. B. Smith, B. D. Auerbach, S. Chattarji, and M. F. Bear, Correction of fragile X syndrome in mice. Neuron, 2007. 56(6): p. 955-62.
7. Chuang, S. C., W. Zhao, R. Bauchwitz, Q. Yan, R. Bianchi, and R. K. Wong, Prolonged epileptiform discharges induced by altered group I metabotropic glutamate receptor-mediated synaptic responses in hippocampal slices of a fragile X mouse model. J Neurosci, 2005. 25(35): p. 8048-55.
8. Yan, Q. J., M. Rammal, M. Tranfaglia, and R. P. Bauchwitz, Suppression of two major Fragile X Syndrome mouse model phenotypes by the mGluR5 antagonist MPEP. Neuropharmacology, 2005. 49(7): p. 1053-66.
9. Lambert, M., P. J. Lupien, C. Gagne, E. Levy, S. Blaichman, S. Langlois, M. Hayden, V. Rose, J. T. Clarke, B. M. Wolfe, C. Clarson, H. Parsons, D. K. Stephure, D. Potvin, and J. Lambert, Treatment of familial hypercholesterolemia in children and adolescents: effect of lovastatin. Canadian Lovastatin in Children Study Group. Pediatrics, 1996. 97(5): p. 619-28.
10. Kobau, R., H. Zahran, D. J. Thurman, M. M. Zack, T. R. Henry, S. C. Schachter, and P. H. Price, Epilepsy surveillance among adults—19 States, Behavioral Risk Factor Surveillance System, 2005. MMWR Surveill Summ, 2008. 57(6): p. 1-20.
11. Mulvihill, B., M. Wingate, R. S. Kirby, S. Pettygrove, C. Cunniff, F. J. Meaney, L. Miller, C. Robinson, G. Quintana, M. Y. Kaiser, L. C. Lee, R. Landa, C. Newschaffer, J. Constantino, R. Fitzgerald, J. Daniels, E. Giarelli, J. Pinto-Martin, S. E. Levy, J. Charles, J. Nicholas, M. Durkin, C. Rice, J. Baio, K. Van Naarden Braun, M. Yeargin-Allsopp, M. Hepburn, N. Garner, K. C. Mancilla, A. Ratchford, Y. Castillo, M. Kolotos, R. Fitzgerald, P. Bell, R. Meade, L. King, C. Arneson, A. Washington, S. Graham, R. Lance, N. Grummon, L. Plummer, L. Jones, J. Wojcik, and N. Doernberg, Prevalence of autism spectrum disorders—Autism and Developmental Disabilities Monitoring Network, United States, 2006. MMWR Surveill Summ, 2009. 58(10): p. 1-20.
12. Kanner, L., Autistic disturbances of affective contact. Nervous Child, 1943. 2: p. 217-250.
13. Musumeci, S. A., R. J. Hagerman, R. Ferri, P. Bosco, B. Dalla Bernardina, C. A. Tassinari, G. B. De Sarro, and M. Elia, Epilepsy and EEG findings in males with fragile X syndrome. Epilepsia, 1999. 40(8): p. 1092-9.
14. Berry-Kravis, E., Epilepsy in fragile X syndrome. Dev Med Child Neurol, 2002. 44(11): p. 724-8.
15. Yan, Q. J., P. K. Asafo-Adjei, H. M. Arnold, R. E. Brown, and R. P. Bauchwitz, A phenotypic and molecular characterization of the fmr1-tm1Cgr fragile X mouse. Genes Brain Behav, 2004. 3(6): p. 337-59.
16. Krueger, D. D. and M. F. Bear, Toward fulfilling the promise of molecular medicine in fragile X syndrome. Annu Rev Med, 2010. 62: p. 411-29.
17. Shaul, Y. D. and R. Seger, The MEK/ERK cascade: from signaling specificity to diverse functions. Biochim Biophys Acta, 2007. 1773(8): p. 1213-26.
18. Mendola, C. E. and J. M. Backer, Lovastatin blocks N-ras oncogene-induced neuronal differentiation. Cell Growth Differ, 1990. 1(10): p. 499-502.
19. Li, W., Y. Cui, S. A. Kushner, R. A. Brown, J. D. Jentsch, P. W. Frankland, T. D. Cannon, and A. J. Silva, The HMG-CoA reductase inhibitor lovastatin reverses the learning and attention deficits in a mouse model of neurofibromatosis type 1. Curr Biol, 2005. 15(21): p. 1961-7.
20. Konstantinopoulos, P. A., M. V. Karamouzis, and A. G. Papavassiliou, Post-translational modifications and regulation of the RAS superfamily of GTPases as anticancer targets. Nat Rev Drug Discov, 2007. 6(7): p. 541-55.
21. Schafer, W. R., R. Kim, R. Sterne, J. Thorner, S. H. Kim, and J. Rine, Genetic and pharmacological suppression of oncogenic mutations in ras genes of yeast and humans. Science, 1989. 245(4916): p. 379-85.
22. Rodenburg, J., M. N. Vissers, M. D. Trip, A. Wiegman, H. D. Bakker, and J. J. Kastelein, The spectrum of statin therapy in hyperlipidemic children. Semin Vasc Med, 2004. 4(4): p. 313-20.
23. Gegg, M. E., R. Harry, D. Hankey, H. Zambarakji, G. Pryce, D. Baker, P. Adamson, V. Calder, and J. Greenwood, Suppression of autoimmune retinal disease by lovastatin does not require Th2 cytokine induction. J Immunol, 2005. 174(4): p. 2327-35.
24. Yamada, T., N. Shinnoh, T. Taniwaki, Y. Ohyagi, H. Asahara, Horiuchi, and J. Kira, Lovastatin does not correct the accumulation of very long-chain fatty acids in tissues of adrenoleukodystrophy protein-deficient mice. J Inherit Metab Dis, 2000. 23(6): p. 607-14.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a subject having a seizure disorder, comprising the step of administering a composition that includes at least one Ras inhibitor.

2. The method of claim 1, wherein the Ras inhibitor includes a farnesyl transferase inhibitor.

3. The method of claim 2, wherein the farnesyl transferase inhibitor includes a 3-hydroxy-3-methylglutaryl-Coenzyme A reductase inhibitor.

4. The method of claim 3, wherein the 3-hydroxy-3-methylglutaryl-Coenzyme A reductase inhibitor includes at least one member selected from the group consisting of Formulas I and II:

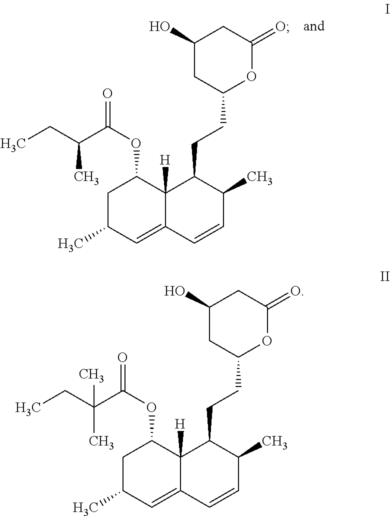 [SMILES: CC[C@H](C)C(=O)O[C@H]1C[C@@H](C)C=C2C=C[C@H](C)[C@H](CC[C@@H]3C[C@@H](O)CC(=O)O3)[C@H]21 ; CCC(C)(C)C(=O)O[C@H]1C[C@@H](C)C=C2C=C[C@H](C)[C@H](CC[C@@H]3C[C@@H](O)CC(=O)O3)[C@H]21]

5. The method of claim 1, wherein the subject has autism spectrum disorder.

6. The method of claim 5, wherein the subject further has fragile X syndrome.

7. The method of claim 1, wherein the subject has fragile X syndrome.

8. The method of claim 1, wherein the Ras inhibitor is administered to the subject in a single dose.

9. The method of claim 1, wherein the Ras inhibitor is administered to the subject in multiple doses.

10. The method of claim 4, wherein the 3-hydroxy-3-methylglutaryl-Coenzyme A reductase inhibitor is administered to the subject in a single dose.

11. The method of claim 4, wherein the 3-hydroxy-3-methylglutaryl-Coenzyme A reductase inhibitor is administered to the subject in multiple doses.

12. The method of claim 4, wherein the 3-hydroxy-3-methylglutaryl-Coenzyme A reductase inhibitor is administered daily in at least one dose selected from the group consisting of a 1 mg dose, 5 mg dose, a 10 mg dose, a 20 mg dose, a 25 mg dose, a 40 mg dose, a 50 mg dose, a 80 mg dose, a 100 mg dose, a 120 mg dose, a 125 mg dose, a 160 mg dose, a 200 mg dose, a 400 mg dose and a 600 mg dose.

13. The method of claim 1, wherein the seizure disorder is an audiogenic seizure disorder.

14. The method of claim 1, wherein the seizure disorder is an epileptic seizure disorder.

15. The method of claim 1, wherein the subject is a human.

16. The method of claim 1, wherein the subject has at least one condition selected from the group consisting of Angelman syndrome, Costello syndrome, cardio facio cutaneous syndrome, neurofibromatosis type I, Noonan syndrome and Coffin-Lowry syndrome.

17. A method of treating a subject having a seizure disorder, comprising the step of administering a composition that includes at least one 3-hydroxy-3-methylglutaryl-Coenzyme A reductase inhibitor.
